SEQUENCE LISTING (4 sequences)

SEQ ID NO: 1 - protein, 480 aa, Homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRLHNGRSKEQRLKEQLGAQQEPVKKSIQESEAFLPQSIPEERYKMKSKPLGICLIIDCIGNETELLRDTFTSLGYEVQKFLHLSMHGISQILGQFACMPEHRDYDSFVCVLVSRGGSQSVYGVDQTHSGLPLHHIRRMFMGDSCPYLAGKPKMFFIQNYVVSEGQLENSSLLEVDGPAMKNVEFKAQKRGLCTVHREADFFWSLCTADMSLLEQSHSSPSLYLQCLSQKLRQERKRPLLDLHIELNGYMYDWNSRVSAKEKYYVWLQHTLRKKLILSYT

SEQ ID NO: 2 - DNA, 2243 bp, Homo sapiens
ggacgtcgaggcattacaatcgcgaaaccaagccatagcatgaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttcctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatttggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggagaacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccgtccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatgcaaaaattagctgggtgtgggtgtgggtacctgtgttcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaa

SEQ ID NO: 3 - protein, 221 aa, Homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRMITPYAHCPDLKILGNCSM

SEQ ID NO: 4 - DNA, 1360 bp, Homo sapiens
cactcgcagtgtttactcctaacgcgtggaacttgtgtcgacatccacccccggttactgcatactcagtcacacaagccatagcaggaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggatgataacaccctatgcccattgtcctgatctgaaaattcttggaaattgttccatgtgattaacatggaactgcctctacttaatcattctgaatgattaaatcgtttcattttctaaatgtgttataatgtgtttagccctttcttgttgctgtatgtttagatgctttccaatcttttgttactactaataatgctataaaataaatatccttgtacttctttga

(19) United States
(12) Patent Application Publication
(10) Pub. No.: US 2012/0015352 A1
(43) Pub. Date: Jan. 19, 2012

(54) METHOD OF DETERMINING SENSITIVITY OF HUMAN OR NON-HUMAN ANIMAL CELLS TO AN IAP ANTAGONIST

(75) Inventors: Martin Leverkus, Madgeburg (DE); Peter Geserick, Madgeburg (DE); Mike Hupe, Madgeburg (DE)

(73) Assignee: Otto-von-Guericke-Universitat Magdeburg, Madgeburg (DE)

(21) Appl. No.: 13/146,817

(22) PCT Filed: Jan. 29, 2010
(86) PCT No.: PCT/IB2010/000165

§ 371 Date: Oct. 10, 2011

Related U.S. Application Data
(60) Provisional application No. 61/148,164, filed on Jan. 29, 2009.

(51) Int. Cl. C12Q 1/68; C12N 5/071; C12N 5/09; C12Q 1/02

(52) U.S. Cl. 435/6.1; 435/29; 435/375

(57) ABSTRACT

cFLIP serves as a biomarker for efficacy of treatment with IAP antagonists, including Smac peptidomimetics.

### CROSS REFERENCE TO RELATED APPLICATION

This application claims the benefit of U.S. Provisional 61/148,164, filed Jan. 29, 2009, the entire disclosure of which is incorporated herein by reference in its entirety.

### FIELD OF THE INVENTION

This invention is in the field of SMAC mimetics and compositions and uses thereof to treat proliferative disorders including cancers.

### BACKGROUND OF THE INVENTION

The development of apoptosis resistance is a mainstay of tumor formation and represents a major obstacle for tumor therapy¹ Novel therapeutic regimen aiming at the reactivation of the apoptotic machinery are intensely studied and, consequently, a variety of compounds that target central molecules within the apoptotic signalling cascades such as death receptor agonists, Bcl-2 antagonists, or inhibitors of the inhibitor-of-apoptosis proteins (IAPs) are currently being explored for their clinical use²⁻⁴.

TNF-related apoptosis-inducing ligand (TRAIL) and CD95L are widely studied death ligands, and numerous studies have investigated the signalling capabilities of these death receptors (for review see 5, 6. In particular TRAIL is considered as a promising ligand enabling the specific elimination of tumor cells^{3,7}. Death receptor signalling pathways are controlled at multiple levels, including the receptor expression on the cell surface, the expression of inhibitors such as cellular FLICE-inhibitory protein (cFLIP), X-linked IAP (XIAP), or Bcl-2 family proteins (e.g. Bcl-2, Bcl-X) (for review see 8). Recent evidence indicates that death receptor triggering induces a primary membrane-associated complex but also a secondary independent signalling platform, similar to the TNF pathway⁹⁻¹¹. The mechanisms leading to formation of these secondary complexes and its critical contribution to apoptosis sensitivity and nonapoptotic signals activated by death receptors has not been elucidated in detail. Further complicating the regulation of the extrinsic apoptosis signalling pathways, additional forms of cell death that do not require activation of caspases have been identified over the past decade such as necrotic as well as autophagic cell death¹². These do not merely represent variations of the cell death pathway but might be particularly relevant in respect to immunological responses within multicellular organisms required for an efficient immune response to cancer cells. Intriguingly, the mode of cell death might define cell death as “immunogenic” versus “silent”, as previously proposed^{13, 14.}

A large body of work over the past decade has revealed that multiple tumors either have or acquire apoptosis resistance during tumorigenesis or by initial treatment, and death receptor agonists alone have not yet yielded encouraging results in early clinical studies¹⁵. IAP antagonists are synthetic compounds that were modeled according to the N-terminal IAP-binding motif (IBM) of the mitochondrial protein Smac/DIABLO to the BIR2/BIR3 domain of XIAP¹⁶. The role of cIAPs for apoptosis resistance to death ligands is less well understood^{17,18}. However the interference with XIAP function is crucial for therapeutic efficiency of TRAIL in xenograft tumor models¹⁹. The use of IAP inhibitors for cancer therapy has been stimulated by the recent independent findings by several groups that IAP inhibitors do not only displace XIAP from binding to effector caspases. Rather IAP inhibitors also induce rapid autoubiquitination and loss of cIAP1 and cIAP2, induction of NF-κB, and autocrine production of TNF that ultimately leads to TNF-mediated caspase-8 activation and cell death²⁰⁻²⁴. In this context is has become clear that cIAP1 and cIAP2 are rather caspase regulators instead of being caspase inhibitors and most likely have additional functions not yet explored in detail.

Cellular inhibitor of apoptosis proteins (cIAPs) are required to protect from TNF-mediated cell death. A role for cIAPs for the sensitivity of tumor cells to prototypical death receptor signalling such as CD95 or TRAIL-R has not been studied in detail.

Cellular-FLICE like inhibitor, cFLIP, is an inhibitor of apoptosis mediated by the death receptors Fas, DR4, and DR5 and is expressed as long (cFLIP_{L}) and short (cFLIP_{S}) splice forms. c-FLIP is an inhibitor of apoptosis mediated by the death receptors Fas, DR4, and DR5 and is expressed as long (c-FLIP_{L}) and short (c-FLIP_{S}) splice forms. cFLIP can inhibit apoptosis mediated by TNF receptor gene superfamily members by interacting with FAS-mediated death domain (FADD) and caspase-8.

Inhibitors of Apoptosis Proteins (IAPs) are naturally occurring intra-cellular proteins that suppress caspase-dependent apoptosis. SMAC, also known as DIABLO, is another intracellular protein that functions to antagonize, i.e., inhibit the activity of IAPs. In normal healthy cells, SMAC and IAPs function together to maintain healthy cells. However, in certain disease states, e.g., cancers and other proliferative disorders, IAPs are not adequately antagonized and therefore prevent apoptosis and cause or exacerbate abnormal proliferation and survival.

SMAC mimetics, also known as IAP antagonists, are synthetic small molecules that mimic the structure and IAP antagonist activity of the four N-terminal amino acids of SMAC. (SMAC mimetics are sometimes referred to as IAP antagonists.) When administered to animals suffering proliferative disorders, the SMAC mimetics antagonize IAPs, causing an increase in apoptosis among abnormally proliferating cells.

Examples of SMAC peptidomimetics are those disclosed in, among others, U.S. Pat. No. 7,517,906; U.S. Pat. No. 7,309,792; U.S. Pat. No. 7,419,975; US 2005/0234042; US 2005/0261203; US 2006/0014700; US 2006/0025347; US 2006/0052311; US 2006/0128632; US 2006/0167066; US 2007/0042428; US 2007/032437; US 2008/0132485; WO 2005/069888; WO 2005/069894; WO 2006/010118; WO 2006/122408; WO 2006/017295; WO 2006/133147; WO 2006/128455; WO 2006/091972; WO 2006/020060; WO 2006/014361; WO 2006/097791; WO 2005/094818; WO 2008/045905; WO 2008/016893; WO 2007/136921; WO 2007/021825; WO 2007/130626; WO 2007/106192; and WO 2007/101347.

### SUMMARY OF THE INVENTION

In one illustrative embodiment, this invention is a biomarker for resistance to induction of apoptosis by an IAP antagonist, i.e., an IAP inhibitor. Specifically, in this embodiment, resistance to treatment with an IAP antagonist is determined by assaying for the long isoform of cFLIP, i.e., cFLIP_{L}. Human or non-human animal cells that express cFLIP_{L} tend to be resistant to IAP antagonists.

In another illustrative embodiment, this invention is a biomarker for sensitivity to induction of apoptosis by an IAP antagonist. Specifically, in this embodiment sensitivity or receptiveness to treatment with an IAP antagonist is determined by assaying for the short isoform of cFLIP, i.e., cFLIP_{S}. Human or non-human animal cells that express cFLIP_{S} tend to be sensitive to IAP antagonists.

In more specific illustrative embodiments, the invention comprises such method wherein sensitivity of the cells to an IAP antagonist in combination with a TRAIL receptor agonist, a CD95 receptor agonist or a TNFa receptor agonist is determined, e.g., such method wherein the TRAIL receptor agonist is TRAIL, the CD95 receptor agonist is CD95L, and the TNFa receptor is TNFã.

In particular illustrative embodiments, the human or non-human animal cells are from a biopsy sample, or a cell line. The cells may be any cells that are proliferating abnormally, e.g., tumor cells or cells that abnormally proliferate in an autoimmune disorder.

In particular illustrative embodiments, the potential for expression of the cFLIP_{L} or the cFLIP_{S} gene in a cell is assayed by:

(a) determining the presence of cFLIP_{L} or cFLIP_{S} mRNA in the cell,

(b) determining the presence of cFLIP_{L} or cFLIP_{S} in the cell.

In another illustrative embodiment, the invention is a method of treating a patient suffering a proliferative disorder that comprises:
(a) determining the sensitivity of proliferative cells to treatment with an IAP antagonist by determining if the cells can express cFLIP_{L} or cFLIP_{S}, whereby cells that can express cFLIP_{S} are sensitive to an IAP antagonist. and
(b) if the cells can express cFLIP_{S}, then treating the cells with an IAP antagonist or
(c) if the cells can express cFLIP_{L}, then treating the cells with an agent other than or in addition to an IAP antagonist or increasing the dose of the IAP antagonist.

While the method of the invention can be carried out directly on the human or animal body, it is not necessary to do so. Rather, the method can be carried out using a sample, such as a biopsy. Thus, in another illustrative embodiment, the invention comprises the use of an agent that detects the presence of cFLIP_{L} or cFLIP_{S}, or of mRNA for cFLIP_{L} or cFLIP_{S}, to treat a patient suffering from a proliferative disorder, or to determine whether or not to treat such patient with an IAP antagonist and, if so, at what dose. Such agent, as described further hereinbelow, can be, e.g., an antibody or a nucleotide probe.

Thus, the invention in other illustrative embodiments also comprises a kit for the practice of the methods of the invention, such kit comprising, e.g., a means for detecting the presence of cFLIP_{L} or cFLIP_{S}, or of mRNA for cFLIP_{L} or cFLIP_{S}, said means being, e.g., an agent that is useful in the detection of cFLIP_{L} or cFLIP_{S}, or of mRNA for cFLIP_{L} or cFLIP_{S}, such as described above and hereinbelow.

### FIGURES

FIG. 1. IAP inhibitor sensitizes SCC and HaCaT to death ligand (DL)-mediated apoptosis independent of autocrine TNF secretion. A). HaCaT, MET1, or A5RT3 cells were either pretreated with 100 nM IAP inhibitor alone or in combination with 10 μg/ml TNFR2-Fc for 30 min and then stimulated with indicated concentrations of TRAIL (ng/ml) or CD95L (U/ml) in triplicate wells. Viability of cells was analyzed by crystal violet assay after 18-24 hrs. Unstimulated cells served as control and were set as 100% to allow comparison of the death ligand-independent sensitivity. The summary of four independent experiments is shown and error bars describe the standard error of mean (SEM). B-E). IAP inhibitor increases CD95L-mediated cell death. HaCaT cells were either prestimulated with IAP inhibitor (100 nM) for 30 min alone or stimulated/costimulated with CD95L (10 U/ml) for 4 hrs (B), 8 hrs (hypodiploidy analysis; 8 hrs), or for the cleavage of caspases or PARP-1 (indicated time periods). B) Cells were stained with Annexin-V-Cy5 and propidium iodide (PI) and then analyzed by FACS. C) Cells were incubated for 8 h, subsequently resuspended in hypotonic buffer including PI (see material and methods) followed by FACS analysis. D) For clonogenic assays, HaCaT cells were prestimulated with IAP inhibitor (100 nM) for 30 min followed by costimulation with CD95L (2.5 U/ml). 24 hrs after stimulation, medium was changed after several washes in sterile PBS, new medium was added and the cells were cultured for another 5 or 7 days followed by crystal violet staining. One representative experiment of a total of three independent experiments is shown. E) For biochemical analysis, HaCaT cells were either treated with IAP inhibitor (100 nM) or CD95L (2.5 U/ml) alone or in combination of both in the presence or absence of TNF-R2-Fc (10 μg/ml) for the indicated time points, and Western blot analysis for the expression of cIAP1, cIAP2, cFLIP, Caspase-8, PARP-1, FADD, and RIP1 was performed. β-Tubulin served as an internal loading control. One of two representative experiments is shown.

FIG. 2. A) HaCaT, A5RT3, and MET1 cells were treated with IAP Antagonist (100 nM) or co-stimulated with TNF-R2-Fc (10μ/ml) for the indicated time. Sufficient decrease of cIAP1 and cIAP2 expression in all cell lines and expression of XIAP and caspase 3 in MET1 cells was controlled by Western blot analysis with specific abs to the respective proteins. β-Tubulin served as internal control. One of four representative results is shown. B) Varying concentration of SMAC mimetic (6-400 nM of IAP) was added to HaCaT, A5RT3, MET1 and SCC25 cell lines to determine cell viability. At the end of incubation, cells were stained with crystal violet and viability was determined. C) Cell lines co-incubated with SMAC mimetic (6-400 nM) and 10 μg/ml TNF-R2-Fc as in C to determine viability with crystal violet. D) HaCaT and MET1 cells were either not stimulated or stimulated with IAP antagonist (100 nM) for 4 h. Surface expression of CD95, TRAIL-R1, and TRAIL-R2 were specifically stained with respective antibodies to death receptors and visualized by FACS. One of two representative results is shown.

FIG. 3. Death receptor-mediated cell death in the presence of IAP inhibitor is neither entirely caspase-dependent nor caspase-independent. Inhibition of caspase activity by unique caspase inhibitor zVAD-fmk partially protects HaCaT cells death ligand-mediated cell death in the presence of IAP inhibitor. A) HaCaT cells were prestimulated or costimulated with zVAD-fink (10μ04; 1 h), necrostatin-1 (50 μM, 1 h), and IAP inhibitor (100 nM, 30 min). Subsequently cells were stimulated with the indicated concentration of TRAIL or CD95L in triplicate wells. Viability of cells was analyzed by crystal violet assay after 18-24 h of incubation as indicated in materials and methods. SEM are shown for 7 independent experiments. B) For analysis of DNA condensation HaCaT cells were either pretreated with zVAD-fink (10 μM, 1 h) or IAP inhibitor (100 nM, 30 min). Cells were subsequently stimulated with CD95L (5 U/ml) for 4 hrs or 24 hrs, respectively. Hoechst-33342 (5 μg/ml) was added for 15 min at 37° C. immediately followed by transmission (left) or fluorescence (right) microscopy. One of two independent experiments is representatively shown.

FIG. 4. RIP1 is an important regulator of death ligand mediated cell death in the absence of cIAPs. A) Endogenous protein expression levels of FADD, cFLIP, Caspase-8, TRAF2, RIP1, cIAP1, cIAP2, and XIAP were analyzed by Western blotting of 5 μg of total cellular lysates of HaCaT, A5RT3, MET1, and SCC25 cells. β-Tubulin served as internal control for even loading. One of three representative results is shown. B) Inhibition of both caspase activity by zVAD-fmk and RIP1 kinase activity by necrostatin-1 completely protects HaCaT cells death ligand-mediated cell death in the presence of IAP inhibitor. HaCaT cells were separately prestimulated with zVAD-fmk (10 μM; 1 h), necrostatin-1 (50 μM, 1 h) and IAP inhibitor (100 nM, 30 min), followed by stimulation with TRAIL (50 ng/ml) or CD95L (2.5 U/ml) in triplicate wells by crystal violet assay. SEM of three (TRAIL) or five (CD95L) independent experiments are shown. C. Stable knockdown of RIP1 protects HaCaT cells from death ligand-induced cell death. HaCaT cells were retrovirally transduced with either hyper random sequence shRNA (HRS) or RIP1-specific-shRNA and selected for 3 days with puromycin (3 μg/ml). Knockdown efficiency of RIP1 was controlled by Western blot analysis for RIP1. Reprobing of the membrane with Abs to β-Tubulin serves as an internal control for protein loading. D) Transduced HaCaT cells as shown in C) were prestimulated for 30 min with 100 nM IAP inhibitor and TNF-R2-Fc (10 μg/ml), and subsequently stimulated with the indicated concentrations of TRAIL or CD95L for 18-24 hrs and assayed by crystal violet assay SEM of three (TRAIL) or four (CD95L) independent experiments are shown. E) Transduced HaCaT as described in C) were preincubated with IAP inhibitor (100 nM) for 30 min followed by stimulation with CD95L (0.5 U/ml). After 24 hrs, culture medium was removed, cells were washed with sterile PBS, and new medium was added. Cells were subsequently cultured for another 5d followed by crystal violet staining. One of four representative independent experiments is shown.

FIG. 5. Induction of Ligand-induced receptor bound CD95 complex (DISC) or intracellular caspase-8-containing complex (complex II) in the presence or absence of IAP inhibitor A) The CD95 DISC was precipitated from MET1 or A5-RT3 cells stimulated with CD95L-Fc for 2 h. Subsequently, the CD95L DISC (left panel) was precipitated using ligand affinity precipitation as detailed in materials and methods. Precipitation of receptor complexes following lysis (−) served as internal specificity control when compared to ligand affinity precipitates (IP; +). Equal amounts of DISC (CD95L-IP) or caspase-8-interacting proteins (complex II) were subsequently analyzed by Western blotting for the indicated molecules. Equal amounts of total cellular lysates (TL) were loaded on the same gels to allow comparison of signal strength between CD95L-IP, complex II, and TL.

FIG. 6. cFLIP is an important regulator of death ligand mediated cell death in the absence of cIAPs. A) A5-RT3 cells were retrovirally transduced with either hyper random sequence shRNA (HRS) or cFLIP-specific-shRNA and selected for 3 days with puromycin (3 μm/ml). Knockdown efficiency of cFLIP_{L} and cFLIP_{S} was controlled by Western blot analysis. Reprobing of the membrane with Abs to RIP1, FADD, Caspase-8, and β-Tubulin serves as an internal control for protein loading. Shown is a representative of three independent experiments. B) Transduced A5-RT3 as shown in A) were prestimulated for 30 min with 100 nM IAP inhibitor and TNF-R2-Fc (10 μg/ml), and subsequently stimulated with the indicated concentrations of TRAIL (left panel) or CD95L (right panel) for 18-24 hrs and assayed by crystal violet assay C) Inhibition of caspase activity (zVAD-fmk; 10 μM) and RIP1 kinase activity by necrostatin-1 (50 μM) completely protects A5-RT3 cells from death ligand-mediated cell death in the presence of IAP inhibitor. Transduced A5-RT3 cells were separately prestimulated with zVAD-fmk (10 μM; 1 h), necrostatin-1 (50 μM, 1 h) and IAP inhibitor (100 nM, 30 min), followed by stimulation with CD95L (25 U/ml) in triplicate wells. Viability of cells was analyzed by crystal violet assay. SEM of four independent experiments are shown. D HaCaT cells were retrovirally transduced with cFLIP_{L} or cFLIP_{S} or control vector. Total cellular lysates were analyzed for cFLIP and caspase-8. β-Tubulin serves as an internal control for protein loading. Comparable results were obtained in 2 additional independent experiments; E) Transduced HaCaT cells as indicated in D) were prestimulated with zVAD-fmk (10 μM; 1 h), necrostatin-1 (50 μM, 1 h), and IAP inhibitor (100 nM, 30 min) or diluent alone. Subsequently cells were stimulated with the CD95L, (25 U/ml) in triplicate wells. Viability of cells was analyzed by crystal violet assay after 18-24 hrs. Shown is SEM of seven independent experiments. F) For clonogenic assays, transduced HaCaT cells were prestimulated with IAP inhibitor (100 nM) for 30 min followed by costimulation with CD95L (2.5 U/ml). 24 hrs after stimulation, medium was changed after several washes with sterile PBS, new medium was added and the cells were cultured for another 5 or 7 days followed by crystal violet staining. One representative experiment of a total of three independent experiments is shown.

FIG. 7. cFLIP_{L}, but not cFLIP_{S} blocks formation of complex II Induction of Ligand-induced receptor bound CD95 complex (DISC) or intracellular caspase-8-containing complex (complex II) in the presence or absence of IAP inhibitor The CD95 DISC was precipitated from HaCaT cells stimulated with CD95L-Fc for 2 h. Subsequently, the CD95L DISC (left panel) was precipitated using ligand affinity precipitation as detailed in materials and methods. Precipitation of receptor complexes following lysis (−) served as internal specificity control when compared to ligand affinity precipitates (IP; +). Equal amounts of DISC (CD95L-IP) or caspase-8-interacting proteins (complex II) were subsequently analyzed by Western blotting for the indicated molecules. Equal amounts of total cellular lysates (TL) were loaded on the same gels to allow comparison of signal strength between IP and TL.

FIG. 8. The role of cIAPs during death receptor-mediated cell death cIAPs block formation of a qualitatively different DISC containing full length RIP1. This signalling platform induces cell death in a caspase-dependent as well as caspase-independent manner. A secondary receptor-independent complex II, which is critical for necrotic cell death, also contains the initiator caspases-8 and -10. Caspase-8 cleavage of RIP1 is one hypothetical mechanism of downregulation of RIP1 within the complex, thereby interfering with RIP1-dependent signalling. Alternatively, RIP1 is only recruited to the DISC when ubiquitinated.

FIG. 9. Knockdown of cFLIP by siRNA sensitizes resistant cells to the combination of Smac mimetics and TNF alpha Cell lines which are resistant to Smac mimetic, TNF alpha and the combination of both are sensitized by siRNA mediated knockdown of cFLIP. A549 and IGROV-1 cells were plated into 24 well plates and allowed to attach overnight. Next day, cells were transfected with 100 nM of either control siRNA or cFLIP targeting siRNA. 48 hrs post transfection, cells were treated with either 100 ng/ml TNF alpha, 100 nM Smac peptidomimetic or the combination of both. After an additional 24 hrs, cells were harvested and stained with FITC labelled AnnexinV and propidium iodide. Apoptosis was quantitated by FACS analysis.

### DETAILED DESCRIPTION OF THE INVENTION

As more fully explained and supported in the attached description of experimental methods and results, this invention provides methods of predicting sensitivity (or resistance) of cells to treatment with antagonists of inhibitor of apoptosis proteins (IAP antagonists), alone, i.e., in monotherapy, or in combination with other anti-proliferative therapies, e.g., co-administration with TRAIL, CD95L, or TNFa or their related agonists. Stated another way, the invention relates to an assay method for determining the susceptibility or receptiveness of a particular proliferative cellular disorder to treatment using IAP antagonists. A cell is sensitive to an IAP antagonist if it undergoes apoptosis in response to the IAP antagonist. Methods of the invention are useful for predicting which cells are more likely to respond to an IAP antagonist by undergoing apoptosis. The methods can be used either in laboratory or clinical settings.

Methods of the invention are particularly useful for screening patients, suffering for example from a proliferative disorder, to identify those who could benefit from administration of an IAP antagonist to treat various benign tumors or malignant tumors (cancer), benign proliferative diseases (e.g., psoriasis, benign prostatic hypertrophy, and restenosis), or autoimmune diseases (e.g., autoimmune proliferative glomerulonephritis, lymphoproliferative autoimmune responses). Cancers which potentially can be treated with IAP antagonists include, but are not limited to, one or more of the following: lung adenocarcinoma, pancreatic-cancer, colon cancer, ovarian cancer, breast cancer, mesothelioma, peripheral neuroma, bladder cancer, glioblastoma, melanoma, adrenocortical carcinoma, AIDS-related lymphoma, anal cancer, bladder cancer, meningioma, glioma, astrocytoma, breast cancer, cervical cancer, chronic myeloproliferative disorders (e.g., chronic lymphocytic leukemia, chronic myelogenous leukemia), colon cancer, endocrine cancers, endometrial cancer, ependymoma, esophageal cancer, Ewing's sarcoma, extracranial germ cell tumors, extragonadal germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, intraocular melanoma, islet cell carcinoma, Kaposi sarcoma, laryngeal cancer, leukemia, acute lymphoblastic leukemia, acute myeloid leukemia, lip cancer, oral cavity cancer, liver cancer, male breast cancer, malignant mesothelioma, medulloblastoma, melanoma, Merkel cell carcinoma, metastatic squamous neck cancer, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, ovarian epithelial cancer, ovarian germ cell tumors, ovarian low malignant potential tumors, pancreatic cancer, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, skin cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

Some methods of the invention involve assaying cells for cFLIP_{L} or cFLIP_{S} gene expression or for the potential for cFLIP_{L} or cFLIP_{S} gene expression. Cells that express the cFLIP_{S} isoform or which have the potential to express the cFLIP_{S} isoform tend to be sensitive to one or more IAP antagonists, i.e., tend to undergo apoptosis when treated with an IAP antagonist. Conversely, cells that express the cFLIP_{L} isoform or which have the potential to express the cFLIP_{L} isoform tend to be less sensitive, i.e., tend to be resistant, to one or more IAP antagonists. cFLIP_{L} or cFLIP_{S} gene expression can be assayed by any means known in the art. In some embodiments gene expression is assayed by detecting cFLIP_{L} or cFLIP_{S} protein of a cell. The amino acid sequences for human cFLIP_{L} and cFLIP_{S} are known (SEQ ID NOS: 1 and 3, respectively). cFLIP_{L} or cFLIP_{S} protein (e.g., secreted, contained within a cell, expressed on a cell surface) can be detected, for example, using various immunoassays (ELISA, Western blot, flow cytometry, radioimmunoassays, etc.). cFLIP_{L} and cFLIP_{S} antibodies are available. See, e.g., Chemicon Cat # AB16963; Enzo Life Sciences Cat # ALX-804-127; Santa Cruz, Cat # SC7108, 7111, 8346, and 7109, which are anti-cFLIP_{L} antibodies. Antibodies that are not specific for the S or L isoforms are also publicly available. One of skill in the art knows how to use such antibodies to identify cFLIP and its respective isoforms. E.g., proteins from a cell lysate can be isolated, e.g., by SDS-PAGE, and then using the anti-cFLIP antibody, e.g., in an ELISA or Western blot, to identify cFLIP_{L} or cFLIP_{S} protein, e.g., based on molecular weight, which is approximately 25-28 KD for cFLIP_{S} and approximately 55 KD for cFLIP_{L}.

In other embodiments gene expression is assayed by detecting cFLIP_{L} or cFLIP_{S} mRNA (e.g., by Northern blot, dot blot, RT-PCR, etc.). The DNA sequence of human cFLIP_{L} and cFLIP_{S} are known (SEQ ID NOS: 2 and 4, respectively). See, e.g., Goto et. al. J. Reproduction and Development, 2004, 50(5) 549-555. The known sequence can be used to prepare probes or one could make degenerate probes based on the known amino acid sequences.

A cell which produces any detectable level of cFLIP_{L} or cFLIP_{S} protein or mRNA is a cell which expresses the cFLIP_{L} or cFLIP_{S} isoform, respectively, although the level of gene expression which can be detected will depend on the assay used.

Any cell type can be assayed for cFLIP_{L} or cFLIP_{S} gene expression. The cells can be primary cells (e.g., cells of a biopsy obtained from a patient) or from cell lines. This invention does not require practice on the human or animal body. Of particular interest are cells which proliferate abnormally, including cells which proliferate pathologically and which cause or lead to disease symptoms. Abnormally proliferating cells occur, for example, in cancer, benign proliferative disorders, and autoimmune diseases.

Cells can be induced to express the cFLIP gene and methods are known to those skilled in the art; selective inducement of cFLIP_{L} or cFLIP_{S} expression, at the level of transcription or translation, can alter phenotype with respect to sensitivity or resistance to IAPs.

Cells expressing cFLIP can be silenced with SiRNA and methods are known to those skilled in the art; selective silencing of cFLIP results in altering the phenotype with respect to sensitivity to IAPs.

Some embodiments of the invention include inducing apoptosis of cells, particularly pathologically proliferating cells. The methods can be carried out in vitro or in vivo and can include treatment of a patient with an IAP antagonist. Such treatment can include administration of a single IAP antagonist, administration of a combination of IAP antagonists, or administration of one or more IAP antagonists and one or more additional chemotherapeutic agents. Administration of multiple agents can be simultaneous or sequential. Useful chemotherapeutic agents include, but are not limited to, alkylating agents (e.g., cyclophosphamide, mechlorethamine, chlorambucil, melphalan), anthracyclines (e.g., daunorubicin, doxorubicin, epirubicin, idarubicin, mitoxantrone, valrubicin), cytoskeletal disruptors (e.g., paclitaxel, docetaxel), epothilones (e.g., epothilone A, epothilone B, epothilone D), inhibitors of topoisomerase II (e.g., etoposide, teniposide, tafluposide), nucleotide analogs precursor analogs (e.g., azacitidine, azathioprine, capecitabine, cytarabine, doxifluridine, fluorouracil, gemcitabine, mercaptopurine, methotrexate, tioguanine), peptide antibiotics (e.g., bleomycin), platinum-based agents (e.g., carboplatin, cisplatin, oxaliplatin), retinoids (e.g., all-trans retinoic acid), and vinca alkaloids and derivatives (e.g., vinblastine, vincristine, vindesine, vinorelbine). In some embodiments, chemotherapeutic agents include fludarabine, doxorubicin, paclitaxel, docetaxel, camptothecin, etoposide, topotecan, irinotecan, cisplatin, carboplatin, oxaliplatin, amsacrine, mitoxantrone, 5-fluoro-uracil, or gemcitabine.

### IAP Antagonists

An IAP antagonist for use in the invention is any molecule which binds to and inhibits the activity of one or more IAPs, such as a cellular IAP (cIAP, e.g., cIAP-1 or cIAP-2) or X-linked IAP(XIAP). In some embodiments, the IAP antagonist preferentially binds XIAP, cIAP-1, or cIAP-2. In some embodiments, the IAP antagonist is a mimetic of Smac (second mitochondrial activator of caspases), and in particular embodiments the Smac mimetic is a mimetic or peptidomimetic of the N-terminal 4-amino acids of mature Smac (Ala-Val-Pro-Ile) or, more generally, Ala-Val-Pro-Xaa, wherein Xaa is Phe, Tyr, Ile, or Val, preferably is Phe or Ile.

In some embodiments of the invention, pharmaceutical compositions comprising an IAP antagonist are administered to a human or veterinary subject. The pharmaceutical compositions typically comprise a pharmaceutically acceptable carrier or diluent and can be administered in the conventional manner by routes including systemic, topical, or oral routes. For example, administration can be, but is not limited to, parenteral, subcutaneous, intravenous, intramuscular, intraperitoneal, transdermal, oral, buccal, intravaginal, or ocular routes, by inhalation, by depot injections, or by implants. Specific modes of administration will depend on the indication and other factors including the particular compound being administered. The amount of compound to be administered is that amount which is therapeutically effective. The dosage to be administered will depend on the characteristics of the subject being treated, e.g., the particular patient treated, age, weight, health, types of concurrent treatment, if any. Frequency of treatments can be easily determined by one of skill in the art (e.g., by the clinician).

Some embodiments of the invention include a kit for performing the evaluation and analysis of cFLIP gene expression. Such kits include, e.g., Qiagen EPITECT(R) Bisulfite Conversion Kit, followed by cFLIP_{L} or cFLIP_{S} sequencing, antibodies, probes, detectable markers and the like, as well as reagents, gels, apparatuses, analysis tools and so forth necessary to perform the evaluation and analysis of IAP antagonist treatment as described above.

The invention includes methods for marketing IAP antagonists, kits, systems, and methods for using biomarkers useful in determining the likelihood of successful treatment using IAP antagonists. In one embodiment, data regarding the effectiveness of such methods, systems and kits is submitted to a regulatory agency as part of a dossier for seeking approval to conduct human clinical trials with an IAP antagonist, e.g., to establish exclusion or inclusion criteria or to facilitate evaluation of clinical trial data. Such data can be submitted to a regulatory agency to support an application for approval to market methods, systems, and kits for using biomarkers associated with treatment using IAP antagonists. For example, such data can be submitted as a part of a New Drug Approval Application (NDA) with the United States Food and Drug Administration (FDA).

Various embodiments of the invention include providing information about the responsiveness of cells that are capable of expressing cFLIP_{L} or cFLIP_{S} in response to treatment with an IAP antagonist and disseminating this information to individuals who may be interested in such a pharmaceutical composition comprising an IAP antagonist. Such individuals include those who have a proliferative disorder, medical personnel who treat such disorders, and individuals who dispense or distribute pharmaceuticals.

When approval has been attained for human clinical trials, the previously described information can be included with data supporting the efficacy of pharmaceutical composition on human subjects exhibiting a proliferative disorder, and other data, such as dosage information and cell toxicity data, in a dossier that can be submitted to a regulatory agency for approval to market an IAP antagonist, and pharmaceutical compositions including the IAP antagonist.

Embodiments also include methods for marketing the IAP antagonist or pharmaceutical compositions including the IAP antagonist after approval has been attained. In such methods, information relating to the fact that IAP antagonists are likely to be effective in cells that are capable of expressing cFLIP_{L} or cFLIP_{S} can be disseminated to, for example, physicians, pharmacists, prescribers, insurance providers, distributors, patients, and the like, or combinations of these. In still other embodiments, the information can be disseminated to prospective patients and/or prospective prescribers, and/or prospective distributors.

The information can be disseminated by any method known in the art including, but not limited to, direct-to-consumer advertising, television advertising, radio advertising, newspaper advertising, advertising through printed materials (e.g., pamphlets, leaflets, postcards, letters, and the like), advertising through a web site or on a web site (using for example, a “banner” ad on a web site), billboard advertising, direct mail, e-mail, oral communications, and any combinations thereof.

In other embodiments, the data can be stored in a user accessible database. The data stored in the database can include any data relating to the IAP antagonist or pharmaceutical composition, including, for example, data generated during testing of the methods, systems, and kits for using biomarkers associated with treatment using IAP antagonists, information regarding safety and/or efficacy of the IAP antagonists, pharmaceutical compositions, methods, systems and kits, dosing information, lists of disorders that can be treated using the compound, approval information from one or more regulatory agency, distributor information, prescription information, and combinations thereof.

Various embodiments also include a system for marketing IAP antagonists, pharmaceutical compositions, methods, systems, and kits for using biomarkers associated with treatment using IAP antagonists including a database, such as the database described above, comprising information regarding the methods, systems and kits and data for the efficacy of methods, systems, and kits for using biomarkers associated with treatment using IAP antagonists. In such embodiments, the information held in the database may only be accessible to selected individuals, such as, for example, management personnel, sales personnel, marketing personnel and combinations thereof. The system can also include a subset of the information held in the database that is disseminated to non-selected individuals who can be any person who is not a selected individual, such as, for example, a physician, a pharmacist, a prescriber, an insurance provider, a patient, a distributor and combinations thereof. Dissemination can take place by any dissemination method known in the art as described above.

The subset of data can include any information held in the database and can include information thought to make the methods, systems, and kits marketable, such as, for example, safety and/or efficacy data, lists of disorders that can be treated using the compound, potential side effects of administering the pharmaceutical, list ingredients or active agents in the pharmaceutical composition, approval information from one or more regulatory agency, distributor information, prescription information and combinations thereof. In certain embodiments, the selected individuals can choose and/or approve the information provided in the subset of data.

In each of the embodiments described above, the information provided and/or disseminated and data stored in the database can further include compositions, methods, or protocols for combined therapies that can include another anti-autoimmune or anti-proliferative agent.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

This invention is more fully described in the attached section, which sets forth experimental methods and data.

### 1. IAP Inhibitor Sensitizes to Death Ligand (DL)-Mediated Cell Death

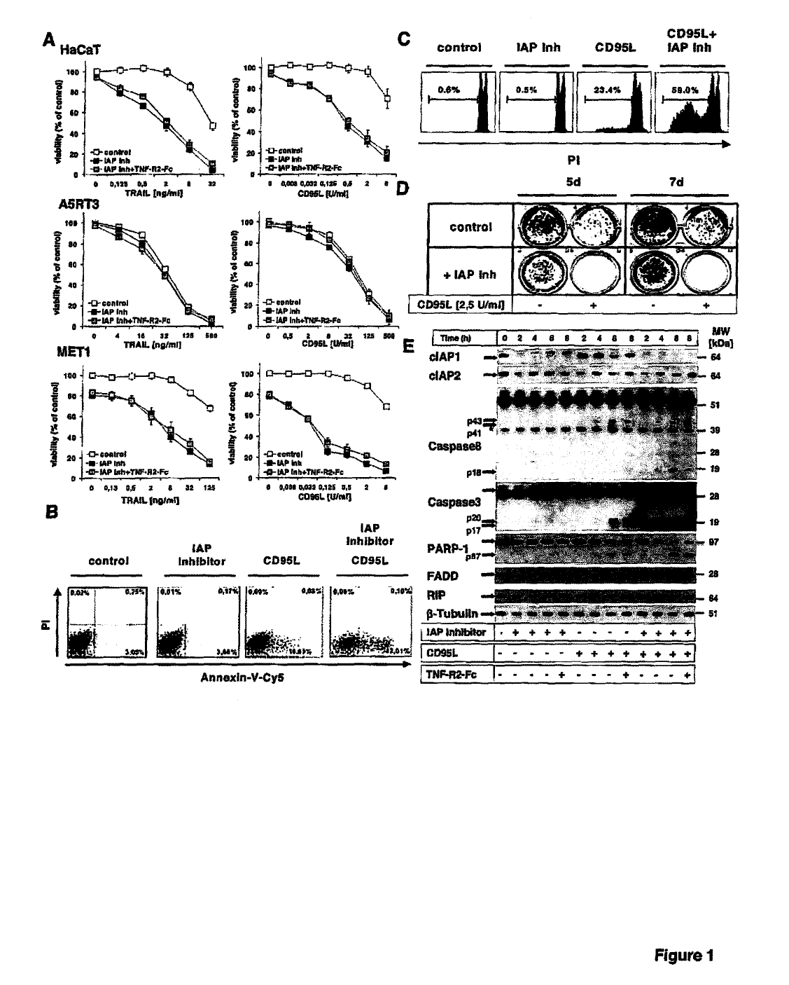

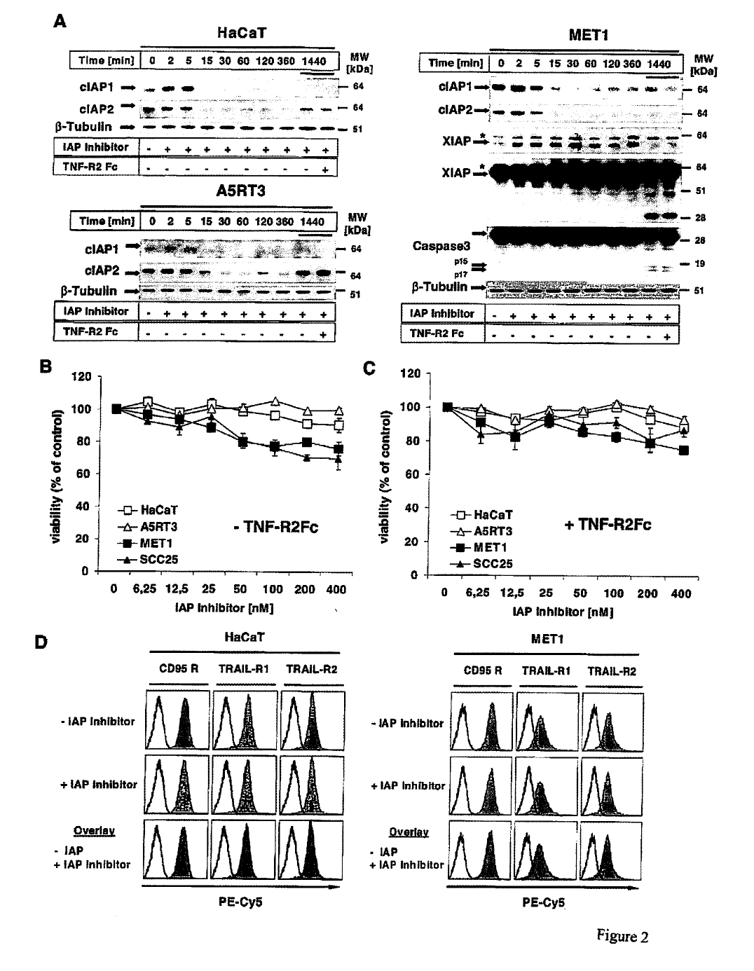

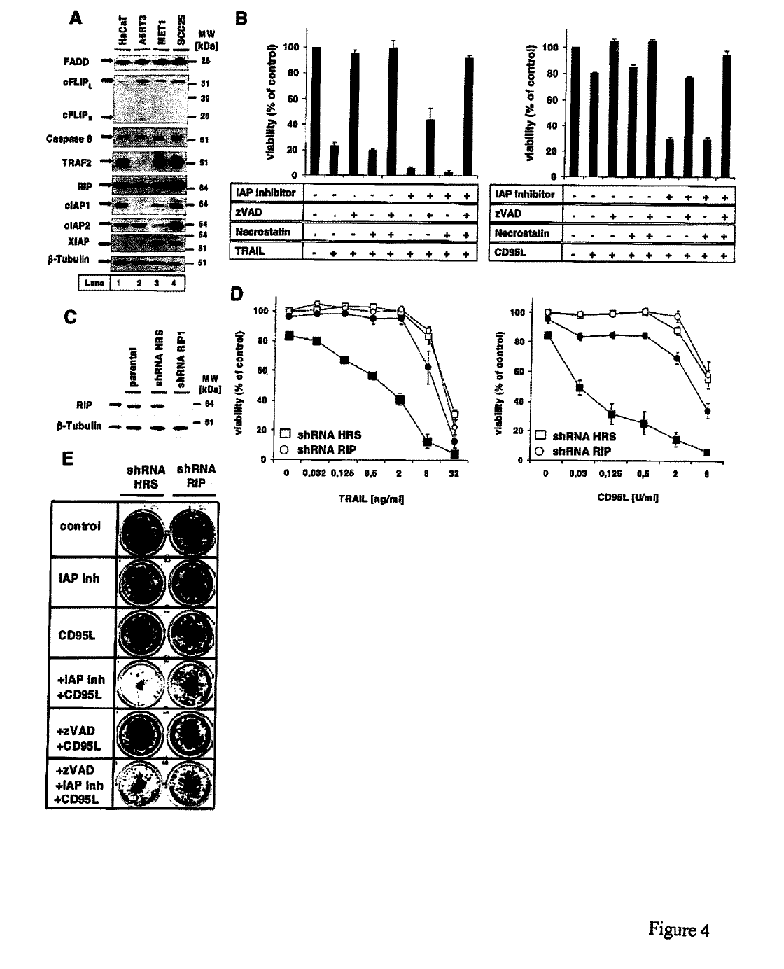

We first characterized the sensitivity of different keratinocyte cell lines and squamous cell carcinoma (SCC) cells with the described IAP inhibitor, Comp. A (IAP inhibitor)²². When cells were incubated for different time periods with 100 nM of this compound, a rapid degradation of cIAP1 as well as cIAP2 was detected (FIG. 2A). Whereas the loss of cIAP1 was persistent throughout 24 hrs, cIAP2 was re-expressed in A5-RT3, and to a lesser extent, in HaCaT cells. In MET1 cells, a caspase-cleaved fragment of 30 kDa of XIAP was detected after 24 hrs irrespective of the inhibition of autocrine TNF signalling using TNF-R2-Fc, whereas HaCaT and A5-RT3 (compare FIG. 4A) do not express XIAP at the protein level^{17,26}. These data indicated that the loss of IAPs induces a rapid degradation of cIAP1 and cIAP2 and lead to caspase-mediated cleavage of XIAP in a TNF-independent manner (FIG. 2A). Dose response studies (6-400 nM of IAP inhibitor) revealed that HaCaT cells as well as the derived metastatic cell line A5-RT3²⁷ were largely resistant to treatment with IAP inhibitor alone up to 400 nM. In contrast, two genetically heterogeneous SCC cell lines (SCC25 as well as MET1) were partially sensitive to the treatment with IAP inhibitor alone (20-25% cell death; Supplemental FIG. 1B). Coincubation with 10 μg/ml TNF-R2-Fc reduced cell death in these cells, indicative of an autocrine loop of TNF production and secretion contributing to cell death in these cells²². We next studied the impact of IAP inhibitor for death ligand sensitivity in these tumor cells. HaCaT as well as MET1 cells, but not A5-RT3 were dramatically sensitized to TRAIL or CD95L-mediated cell death in a TNF-independent manner (FIG. 1A). Cell death occurred by apoptosis as determined by Annexin-V externalization after 4 hrs (FIG. 1B) or hypodiploidy analysis after 8 hrs of incubation with the death ligands (FIG. 1C). Moreover, incubation with IAP inhibitor decreased clonogenic survival (FIG. 1D). When we examined the activation of caspases following treatment with TRAIL or CD95L, we found an increased activation of initiator caspase-8 and effector caspase-3 within 4 hrs after stimulation with the respective death ligands in the presence of IAP inhibitor, resulting in increased cleavage of the effector caspase target PARP (FIG. 1E). In order to confirm these data in an independent experimental system, we used MEF lacking cIAP1. In line with the data in human cells using IAP inhibitor, cIAP1 knockout MEF were sensitized to CD95-mediated cell death. Moreover, inducible reconstitution of cIAP1 into cIAP1 MEF fully restored resistance to the death ligand. Taken together, our data demonstrate that cellular levels of cIAPs are crucial for the maintenance of CD95L or TRAIL resistance.

### 2. Death Receptor-Mediated Cell Death in the Presence of IAP Inhibitor is Neither Entirely Caspase-Dependent Nor Caspase-Independent and Requires RIP1

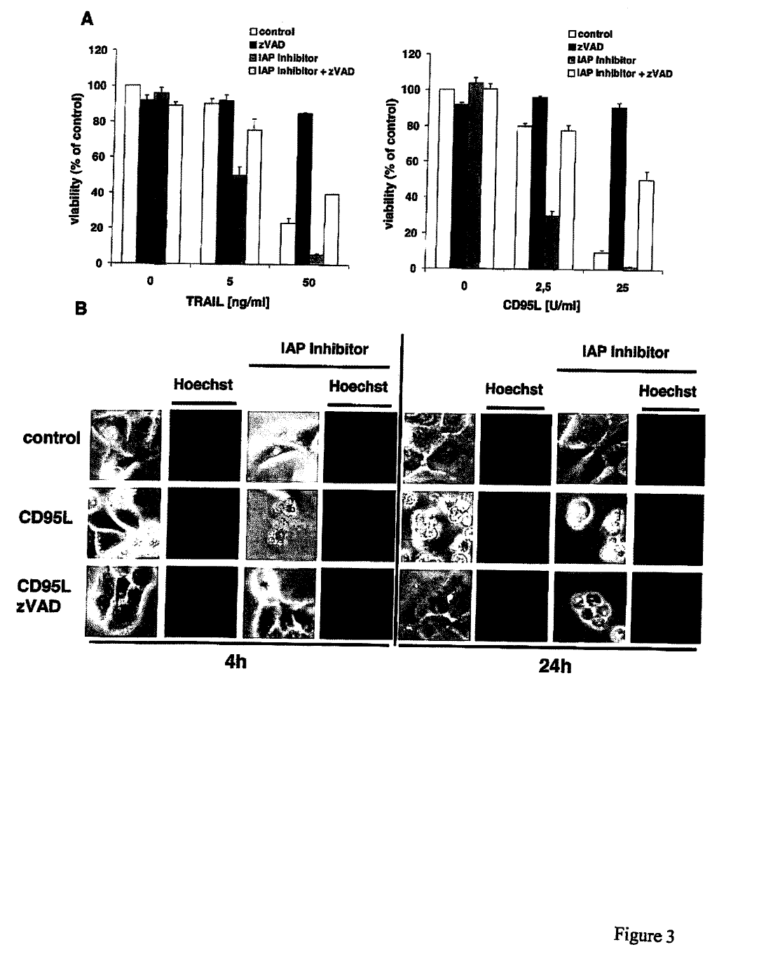

Death receptor-mediated apoptosis is initiated by DISC-activated caspase-8^{6,28}. To investigate if caspase activation is crucial for death receptor-mediated cell death in the presence of IAP inhibitor we next investigated cell death in the presence of the broad spectrum caspase inhibitor zVAD-fmk. As reported by many groups, zVAD-fmk fully blocked cell death when cells were stimulated with TRAIL or CD95L for 24 hrs. However, TRAIL- or CD95L-mediated cell death in the presence of IAP inhibitor was only partially blocked by zVAD-fmk at different concentrations of the death ligands (FIG. 3 A). These data suggested that a caspase-independent form of cell death is induced by death receptor stimulation in the presence of IAP inhibitor. To further characterize the morphologic characteristics of cell death in these cells, we performed fluorescence microscopy studies. Increased numbers of typical apoptotic cells demonstrating membrane blebbing, DNA condensation and fragmentation were detectable only in cells treated in the absence of the caspase inhibitor within 4 hrs after CD95L treatment (FIG. 3 B, left panel). At these early time points, zVAD-fmk fully protected cellular morphology or DNA fragmentation also in the presence of IAP inhibitor. However, zVAD-fmk did not protect cells from cell death 24 hrs after treatment, whereas zVAD-fmk achieved complete protection in the presence of IAPs (FIG. 3B, right panel). These data indicated that IAPs are able to protect from a caspase-independent delayed form of cell death induced by death receptor stimulation. Previous studies have revealed that CD95 activates a caspase-independent form of cell death via the kinase RIP1, although the downstream targets of RIP1 are still unknown²⁹⁻³¹. Since IAPs are able to interfere with ubiquitination of RIP1²⁴, we next investigated the role of RIP1 in our experimental system. Interestingly, RIP1 levels were lowest in A5-RT3 cells that proved to be highly resistant to the sensitizing effect of IAP inhibitor (FIG. 4 A). Necrostatins such as necrostatin-1 have been shown to specifically block the kinase activity of RIP1³². Whereas caspase inhibition only partially protected from TRAIL- or CD95L-mediated cell death, addition of necrostatin-1 to zVAD-fink-protected cells fully recovered viability in the absence of cIAPs. In contrast, necrostatin-1 alone was ineffective to protect against death ligand-mediated cell death in the absence of caspase inhibitor (FIG. 4B). These data hinted at the fact that loss of cIAPs may unmask a death receptor-mediated signal that can only be blocked by combined inhibition of caspases and RIP1. In order to address the role of RIP1 more directly, we generated cell lines with decreased levels of RIP1 using stable shRNA expression (FIG. 4C). Whereas control-infected cells were sensitized to TRAIL or CD95L-mediated cell death by IAP inhibitor this sensitization was largely abrogated in RIP1-repressed cells as determined by viability assays (FIG. 4D) as well as clonogenic assays (FIG. 4E). Collectively, these data indicate that CD95 as well as TRAIL-mediated cell death utilizes a RIP1-dependent signalling pathway that is essential for cell death whenever cIAPs were repressed. To further support the knockdown data, we compared RIP1 knockout MEF with their wild type control cells. RIP1 deficient cells were less sensitive to the induction of TRAIL or CD95L-mediated cell death in the absence of cIAPs. Taken together, these data demonstrate that RIP1 is critically involved in death receptor-mediated cell death in the absence of IAPs.

### 3. IAPs Negatively Regulate the Recruitment of RIP1 to the DISC and Allow for an Increased Formation of a Receptor-Independent Complex II

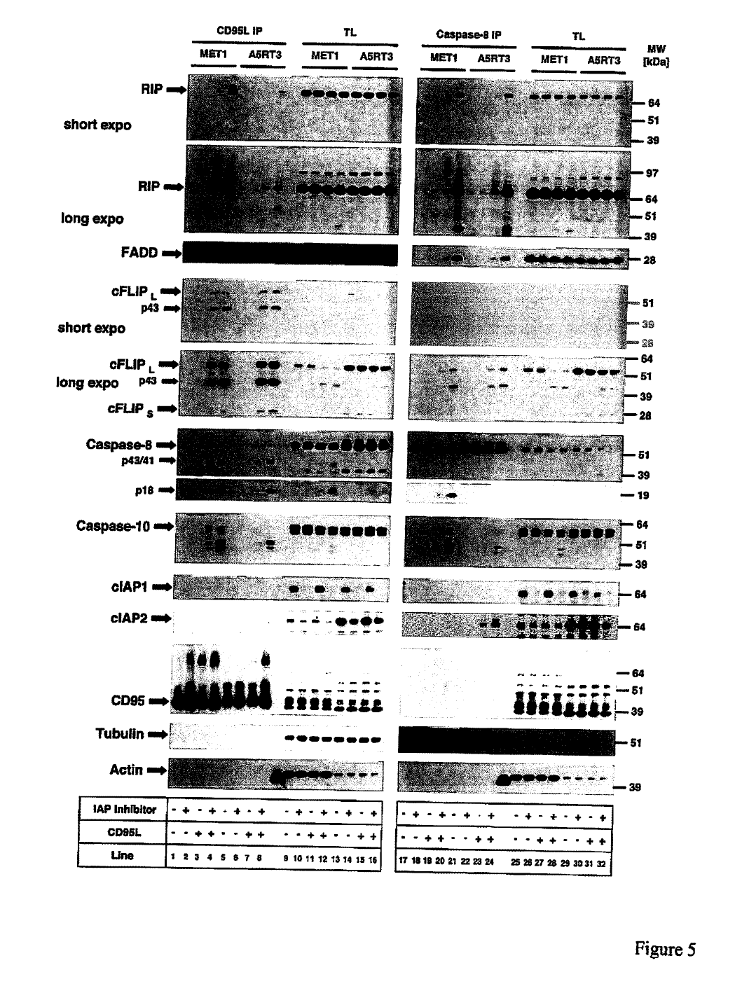

To characterize the molecular mechanism how cIAPs negatively regulate death receptor-mediated cell death, we next characterized components and formation of the native death inducing signalling complex (DISC) in our experimental system. Moreover the recently described formation of a receptor-independent complex II¹⁰ was studied in the absence of IAPs. We chose to analyze the CD95L-induced complexes in two of the cell lines that were either responsive to the sensitizing effect of IAP inhibitor (MET1) and compared it to cells that were not sensitized by loss of IAPs (A5-RT3). Initial experiments revealed that IAP inhibitor did not impact death receptor expression (FIG. 2D and data not shown). We first confirmed the absence of CD95 in the caspase-8-associated complex (designated as complex II) when ligand-affinity precipitation (DISC) and caspase-8-coimmunoprecipitates (complex II) were compared to identical quantities of total cellular lysates of the respective conditions (FIG. 5, TL). In line with our previous data for the TRAIL DISC in HaCaT keratinocytes³³ and other cell types³⁴ and in line with the overall sensitivity to the death ligands (compare FIG. 1), we detected a stimulation-dependent recruitment of cFLIP, caspase-8, caspase-10, FADD, and RIP1 to the CD95 DISC in both cell types (FIG. 5, left panel, lane 3-4, 7-8). Remarkably, despite the higher expression of caspase-8, DISC association of caspase-8 was weaker in A5-RT3, potentially explained by the lower level of CD95 surface expression as determined by western blotting for CD95 (left and right panel, CD95). Comparing the DISC of these two cell lines, A5-RT3 cells demonstrated a comparable recruitment of cFLIP, whereas the recruitment of RIP1, FADD, caspase-8, and caspase-10 was lower in A5-RT3 (compare FIG. 5, left panel, lane 1-4; 5-8). Surprisingly, RIP1 recruitment was strongly increased in the CD95 DISC in MET1 cells as well as A5-RT3 in the absence of cIAPs (FIG. 5, left panel, lane 3-4). In contrast an increased recruitment of RIP1 was detectable, but substantially weaker in A5-RT3 cells (FIG. 5, left panel, lane 7-8). Moreover, we detected a specific stimulation-dependent recruitment of cIAP2 in A5-RT3, but not MET1 cells (FIG. 5, left panel, lane 3-4, 7-8). Examining the interaction of the components of the DISC in a receptor-independent manner by coimmunoprecipitation of caspase-8-associated proteins (complex II), we detected a stimulation-dependent interaction of FADD, caspase-10, cFLIP, and RIP1 with caspase-8 in both cell lines. Initial results had confirmed that the DISC as well as complex II was strongly stabilized in the presence of caspase inhibitors (data not shown). We thus performed these experiments in the presence of zVAD-fmk during stimulation that may alter the extent of caspase-dependent cleavage detectable in our experiments. Interestingly, a large increase of RIP1, FADD, cFLIP_{L} and cFLIP_{L} p43 was detected in association with caspase-8 (FIG. 5, right panel, lane 19-20, 23-24). These data suggested that upon repression of cIAP expression, complex II is increasingly formed in the cytoplasm (FIG. 5, right panel). Interestingly, we found cIAP2, but not cIAP1 in the complex II of A5-RT3, potentially caused by the lower expression level of cIAP2 in MET1 cells (compare FIG. 4 A). Taken together, our data suggested that loss of cIAPs facilitates the recruitment of RIP1 to the CD95 DISC, allows for an increased formation of complex II that contains RIP1-FADD-Caspase-10 and Caspase-8. Moreover, cells resistant to the sensitizing effect of IAP inhibitor demonstrated the presence of cIAP2 within complex II despite treatment with IAP inhibitor, suggesting that cIAP2 might be sufficient to block further activation of the cell death pathway in the presence of IAP inhibitor (and thus loss of cIAP1).

4. cFLIP Isoforms Differentially Contribute to Resistance to Death Ligand-Mediated Cell Death in the Absence of IAPs

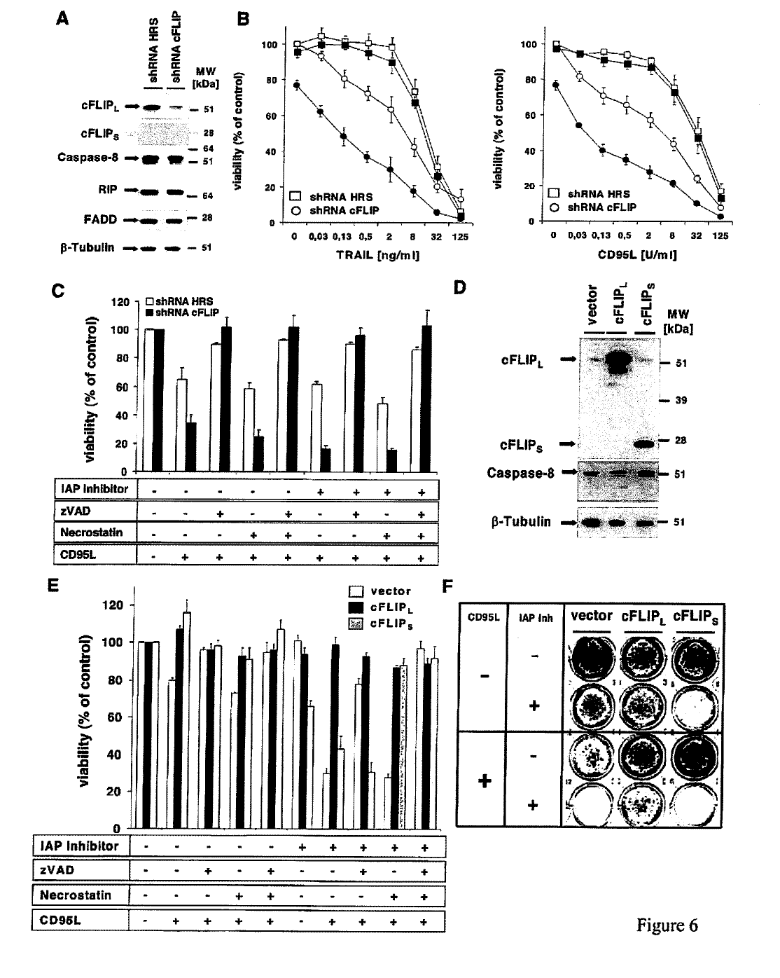

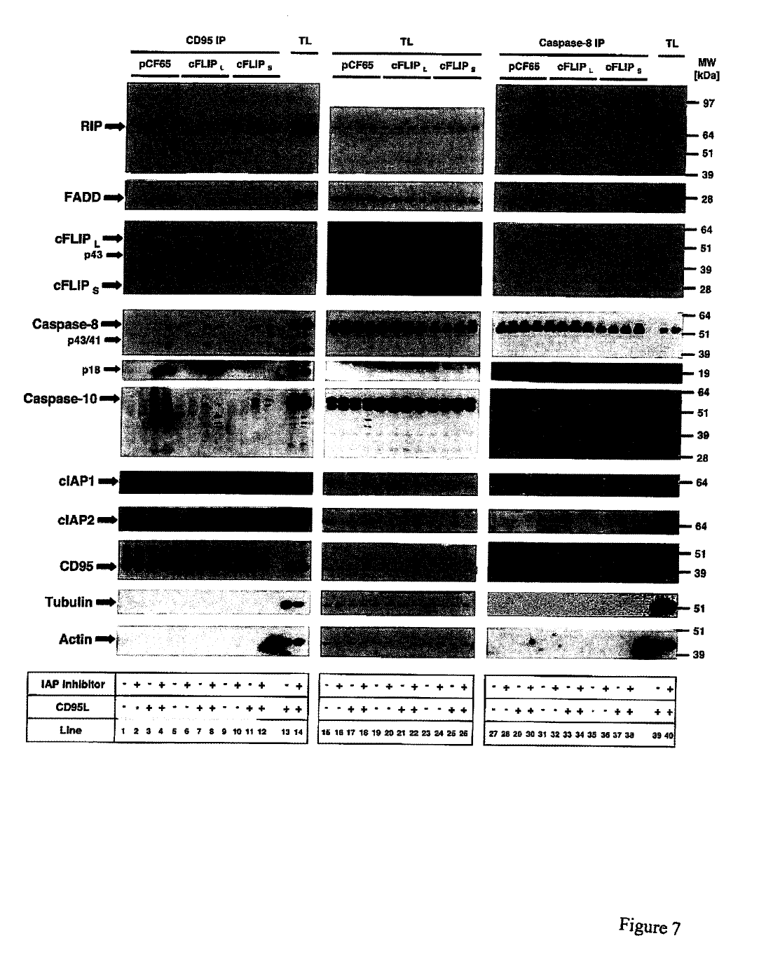

Cells that were sensitive to IAP inhibitor-mediated sensitization to death receptor-mediated cell death recruited to and cleaved caspase-8 within the DISC with high efficiency, whereas recruitment of cFLIP was comparable, indicative of the high affinity of different cFLIP isoforms to the DISC, as previously suggested³³. In this context it is widely accepted that cFLIP is one critical determinant of TRAIL or CD95L cell death resistance (for review see³⁵). We thus next tested, if cFLIP contributes to resistance to IAP inhibitor-mediated sensitization to death ligands. To this end, we chose the primarily IAP-inhibitor resistant SCC cell line A5-RT3 that lacks XIAP and downregulated cFLIP by stable expression of shRNA against cFLIP. Western blot analysis confirmed efficient downregulation of both cFLIP_{L} and cFLIP_{S} (FIG. 6A). Interestingly, a loss of cFLIP in A5-RT3 cells resulted in an increased TRAIL- or CD95L-mediated cell death in the presence of IAP inhibitor (FIG. 6B). In contrast to the data in HaCaT cells, however, sensitization by loss of IAPs was fully caspase-dependent, whereas necrostatin-1 was ineffective in these cells (FIG. 6C). These data confirmed a highly cell-type specific IAP regulation of sensitivity to death ligand-mediated cell death. More importantly, our data indicated that RIP1 as well as cFLIP are critical for this regulation and that in A5-RT3, cFLIP isoforms are able to block IAP inhibitor-mediated cell death. There are a number of conflicting results for the function of different cFLIP isoforms as well as cleavage fragments of cFLIP_{L} for cFLIP's signalling capabilities (for review see³⁶). To address the mechanism of IAP inhibitor-mediated sensitization for death receptor mediated cell death and the impact of cFLIP more specifically, we generated stable cell lines expressing different cFLIP isoforms (FIG. 6D). For these experiments we chose HaCaT keratinocytes due to their low level of endogenous cFLIP³⁷ and their lack of XIAP at the protein level¹⁷. As anticipated, cFLIP_{L} as well as cFLIP_{S} efficiently blocked CD95L- or TRAIL-mediated cell death (FIG. 6 E, panel 2), death receptor-mediated Annexin-V externalization, and DNA hypodiploidy (data not shown). Interestingly, the sensitivity to IAP inhibitor alone was strongly increased in cells expressing cFLIP_{S}, but not cFLIP_{L} (FIG. 7 E, panel 6). Intriguingly, cFLIP_{S} was unable to protect from death receptor-mediated cell death in the presence of IAP inhibitor (FIG. 6 E, panel 7), that was not protected by zVAD-fmk (FIG. 6E, panel 8). In line with our data depicted in FIG. 3, however, both cFLIP isoforms blocked early characteristics of apoptotic cell death (data not shown). However, CD95L-mediated cell death in the presence of IAP inhibitor in cFLIP_{S}-expressing HaCaT was fully protected by necrostatin-1 for 24 hrs, indicative of the contribution of RIP1-dependent signalling (FIG. 6E, panel 9). Taken together our data suggest that cFLIP_{L}, but not cFLIP_{S} is able to block a caspase-independent form of cell death that is activated by death receptors via RIP1 recruitment.

5. cFLIP Isoforms Differentially Influence CD95-Induced Recruitment of RIP1 to Complex II

In order to clarify the potential mechanism of the phenomenon, we next precipitated the CD95 DISC as well as complex II in cFLIP_{L} and cFLIP_{S}-expressing HaCaT cells and compared the results within control-infected HaCaT. Initial results had confirmed that the DISC as well as complex II is strongly stabilized in the presence of caspase inhibitors (data not shown). Since caspase inhibition was not sufficient for protection from cell death, we hypothesized that the presence of zVAD-fmk would thus still allow to monitor differences in these death ligand-induced complexes. We thus stimulated HaCaT expressing the different cFLIP isoforms cells in the presence of zVAD-fink with CD95L in the presence or absence of IAP inhibitor. In line with our data shown in FIG. 5 for A5-RT3 and MET-1, a dramatic increase of RIP1 was detected in the DISC of control cells when cIAPs were lacking (FIG. 7, left panel, lanes 1-4). Consistent with our previous report for the TRAIL DISC³³, cFLIP_{L} as well as cFLIP_{S} repressed the recruitment of RIP1 to the CD95 DISC. Compatible with a previous report in lymphoma cells, cFLIP_{L} led to an increased recruitment of caspase-8 p43/41, whereas cFLIP_{S} fully blocked caspase-8 cleavage in the DISC³⁸. In complex II, there was a substantial increase of RIP1, FADD, and cFLIP_{L} (proform as well as p43) that was coimmunoprecipitated with caspase-8 (FIG. 7, right panel, lane 1-4). In contrast, cFLIP_{L} blocked the formation of complex II. Interestingly, we reproducibly detected complex II formation of cFLIP_{S}-expressing cells in the absence of death ligand stimulation (FIG. 7, right panel, lane 6), indicative of a spontaneous formation of complex II in the absence of cIAPs in these cells. Moreover, the CD95L-stimulated formation of complex II was much stronger when compared to complex II in the presence of cFLIP_{L}, thus providing an explanation for the increased cell death in response to CD95L whenever cIAPs are absent in cFLIP_{S} cells.

### DISCUSSION

In the current invention, we have investigated the mechanism of death receptor-mediated cell death in the context of IAP inhibition. We show that IAP inhibitor dramatically sensitizes SCC cells to DR-mediated cell death largely independent of autocrine TNF inhibition. Instead, IAP inhibitors increase both caspase-8- and RIP1-dependent forms of cell death. To our surprise, different cFLIP isoforms have distinct inhibitory capacities depending on the presence of IAPs. Whereas cFLIP_{L} and cFLIP_{S} similarly inhibit death receptor-mediated apoptosis in the presence of IAPs, cFLIP_{L} blocks RIP1-dependent as well as caspase-8-dependent cell death, and cFLIP_{S} only interferes with caspase-8-dependent apoptosis but was remarkably inefficient in the protection of RIP1-dependent cell death. Our data show for the first time that different cFLIP isoforms have distinct signalling capabilities that are evident only in the absence of cIAPs. This function of cIAPs might not only be relevant for apoptosis resistance as an obstacle of tumor therapy, but be pertinent during virus infection or tumor immunity where the mode of cell death is of paramount importance²⁵.

This invention contributes a number of important findings for the understanding of signalling pathways activated by TRAIL-R1, TRAIL-R2, and CD95 death receptors. First, we unexpectedly find that—in the notable absence of modulations of the death receptor on the cellular surface (data not shown)—loss of cIAPs leads to a dramatic sensitization to TRAIL or CD95L-induced cell death. We studied these aspects in either human SCC tumor cells treated with a pharmacological inhibitor that induces degradation of IAPs within minutes (IAP inhibitor) or alternatively using a genetic model of MEF lacking different cIAPs. Our data demonstrate that TRAIL or CD95L signalling pathways are profoundly regulated by cIAPs largely independent of the suggested autocrine loop of TNF signalling, indicative of an independent function of cIAPs in the death receptor signalling pathway²⁰⁻²⁴ Importantly, our data suggest a molecular mechanism that is operative independent of the function of XIAP, because some of the cells used completely lack XIAP at the protein level¹⁷. Sensitization to IAP inhibitor was also obtained in XIAP MEF further supporting this novel function of cIAPs for the regulation of CD95 and TRAIL-R-mediated cell death.

When we studied the molecular mechanism of this phenomenon, we identified the kinase RIP1 as critical for the negative regulatory role of cIAPs in death receptor-mediated cell death. RIP1 is known for many years for its relevance in NF-κB activation by death receptors³⁹. However the ability of RIP1 to block cell death was considered indirect and mediated by the loss of death receptor-induced NF-κB activation. Other groups had previously investigated programmed necrosis in response to TNF or CD95 stimulation⁴⁰.

Overexpression studies suggested that the DD of FADD is required for necrosis induction whereas the DED of FADD is needed for caspase activation⁴¹. Moreover Holler et al showed CD95-induced necrosis in FADD-deficient Jurkat T cells²⁹. Thus although FADD is involved in both apoptotic as well as necrotic cell death pathways after death receptor triggering, it is unclear if FADD represents the crucial molecular switch for these two signalling pathways. The reason for necrotic cell death in cells expressing FADD and RIP1 remained obscure, although acidic extracellular conditions favor RIP1-dependent cell death, at least in response to TRAIL⁴². Our data now suggest that cIAPs negatively regulate the necrotic cell death pathway, and that RIP1 is necessary for CD95 or TRAIL-R-induced cell death at the level of the DISC. This conclusion is based upon our data using RIP1 knockdown, RIP1-deficient MEF, and the precipitation of the native CD95 complex in tumor cells that contains large amounts of RIP1 in the absence of cIAPs. Furthermore, our data demonstrate that the recruitment of RIP1 to the CD95 membrane-bound complex (CD95 DISC) is dramatically decreased by cIAPs, while the total cellular levels of RIP1 are unaffected. The specific RIP1 kinase inhibitor necrostatin-1 allowed us to further investigate the requirement for RIP1 kinase activity³². Our data clearly show that whenever caspases are blocked, RIP1 kinase becomes a critical protein, and dual inhibition of caspases and RIP1 kinase allows the recovery of cellular viability of CD95- or TRAIL-mediated cell death. We detect a robust enrichment of the DD-containing fragment of RIP1 in the DISC even in the presence of the pancaspase inhibitor zVAD-fmk (data not shown). Unfortunately, all antibodies available thus far recognize epitopes in the DD that precludes detection of the N-terminal fragment that contains the kinase domain of RIP1. Future studies, using tagged proteins, or using antibodies to the kinase domain will further elucidate if cleavage of RIP1 leads to a) a release of the kinase activity from inhibition by the DD in order to induce cell death, or b) is an effective mechanism to remove functional RIP1 altogether.

We find that a loss of IAPs leads to a dramatic sensitization to CD95L or TRAIL in a RIP1-dependent manner. We detect a caspase-8-FADD-cFLIP-RIP1-containing cytoplasmic complex (complex II) that is no longer bound to the death receptor. Formation of this complex is inhibited by cIAPs. Surprisingly, different isoforms of the caspase-8 inhibitor cFLIP have differential effects whenever cIAPs are absent. While all cFLIP_{L} isoforms protect from cell death in the presence of cIAPs, only cFLIP_{L}, but not cFLIP_{S}, is able to block formation of the native complex II, allowing for efficient cell death in the absence of cIAPs. Our data thus identify an important intracellular protein complex relevant for the cell death signalling downstream of CD95 or TRAIL death receptors.

Another important finding of our study is the identification of a receptor-independent complex (complex II) that contains at least FADD, Caspase-8, Caspase-10, RIP1, and different isoforms of cFLIP. A recent report has identified such a complex following CD95 or TRAIL stimulation^{10, 11}. In our experiments the formation of complex II and the association of caspase-8 with RIP1 is repressed in cells insensitive to IAP inhibitor. Remarkably, in these cells cIAP2 is highly expressed, and rapidly reexpressed to steady state levels of untreated cells when examined kinetically after IAP inhibition (Supplemental FIG. 1A). This pattern of cIAP2 expression coincided with detection of cIAP2 associated with the DISC as well as a decreased detection of RIP1 in complex II of these cells (compare FIG. 5) and suggest that cIAP1 and cIAP2 have a differential sensitivity to inhibition by IAP inhibitor or might be differentially regulated altogether. It is perceivable that autocrine TNF secretion may lead to de novo expression of cIAP2. In this context, whereas cIAP1 is repressed by IAP inhibitor in a prolonged manner in our cellular model, cIAP2 is strongly induced by NF-κB activation, although dispensible for the regulation of TNF-mediated cell death¹⁷. Thus both cIAPs might have distinct roles, with cIAP1 being a rather constitutively expressed cIAP, whereas cIAP2 in the inducible form of these highly similar cIAPs with therefore differential functions during distinct pathophysiological processes.

In order to further dissect the function of DISC or complex II-associated signals critical for cell death we performed experiments using the endogenous caspase-8 homologue cFLIP. We demonstrate that cells that are insensitive to the IAP inhibitor (e.g. have overlapping dose response curves) can be sensitized by specific downregulation of cFLIP isoforms. Importantly, these cells are still fully protected by zVAD-fmk, indicative that IAPs also block caspase-dependent signalling pathways, as shown for TNF signalling⁴³. What is the relevance of caspase activation for the activation of the RIP1-dependent cell death within the DISC? We compared the amount of RIP1 recruited to the CD95 complex in the absence and presence of zVAD-fmk and found a marked increase of full length RIP1 in the native CD95 DISC (data not shown) in the presence of zVAD-fmk, in line with our previous report for the TRAIL DISC³³. However zVAD-fink is unable to fully block DISC-associated activity of caspase-8, based upon the cleavage of cFLIP_{L} in the DISC³³. In addition zVAD-fmk does not block the enzymatic activity of the proform of caspase-8⁴⁴. Thus our experiments indicate that cFLIP antagonizes the signal generated by the DISC needed for caspase-dependent as well as caspase-independent cell death. In line, our overexpression studies suggest that cFLIP has a dual role: whereas all isoforms of cFLIP block death receptor-mediated apoptosis with comparable efficiency whenever cIAPs are present, only cFLIP_{L}, but not cFLIP_{S} fully blocks death receptor-triggered cell death in the absence of cIAPs. Importantly, RIP1 kinase activity is critical for the protection of cells from death receptor-mediated cell death in cFLIP_{S}-expressing cells and we detect an increased spontaneous as well as induced formation of complex II with increased levels of FADD and RIP1 in these cells. These data argue that cFLIP_{S} is unable to block the formation of complex II that is negatively regulated by the caspase-like domain of cFLIP_{L}. It is not likely that cFLIP p22 accounts for this differential effect, because this fragment can be generated from cFLIP_{L} as well as cFLIP_{S}⁴⁵. The caspase-like domain of cFLIP_{L} was reported to mediate binding to proteins such as TRAFs, RIP1, or others, mostly based upon overexpression studies and endogenous TRAF2 interacts with DISC-generated cFLIP_{L} p43 (for review see⁴⁶). Moreover, TRAF2 is a binding partner of cIAPs as well as RIP1. However, we were unable to detect TRAF2 in our DISC ligand affinity precipitations or complex II co-immunoprecipiations, respectively (data not shown). Thus, further studies are required to elucidate the role of these additional interacting proteins in more detail.

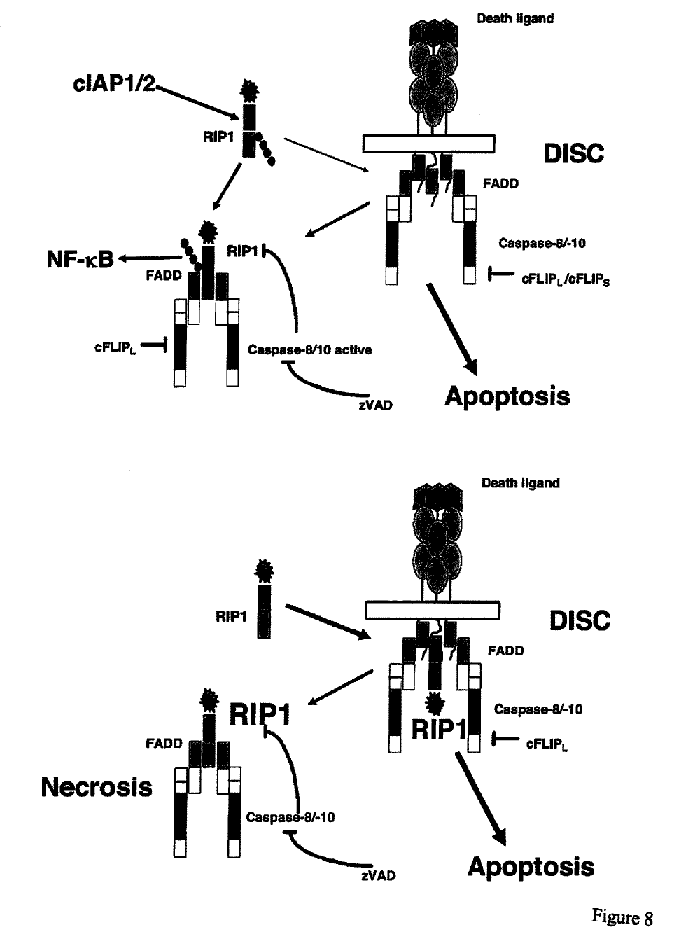

Our data using a number of different SCC cell lines argue that the stoichiometry of different DISC components and their modification by IAPs is highly relevant for the activation of apoptotic as well as necrotic cell death pathways. Using native ligand affinity precipitation of the CD95 DISC we show that cIAPs negatively regulate the amount of RIP1 recruited to the DISC, whereas the total cellular levels of RIP1 are unaltered (compare FIG. 5, 7). RIP1 gains critical relevance once the propagation of death receptor-mediated caspase activation (as studied by pharmacological caspase inhibitors) is blocked and argues for a parallel activation of caspase-dependent as well as caspase-independent cell death at the level of the DISC. Supporting this concept, Holler et al showed recruitment of RIP1 to the DISC in the absence of FADD using FADD-deficient Jurkat cells²⁹. We thus propose that RIP1 constitutes a critical component of a FADD independent signalling pathway that is activated by TRAIL and CD95 death receptors at the DISC and negatively regulated by cIAPs. DISC-associated caspase may act to downregulate RIP1 available in the DISC and suggest that a) cIAP-mediated ubiquitination or b) caspase-mediated cleavage of RIP1 in the DISC represent crucial negative regulatory mechanisms of DISC-activated RIP1-dependent cell death signalling pathways (compare FIG. 8). Future studies using cells deficient in FADD, RIP1, or Caspase-8 will further elucidate the requirements for caspase activity as a potential destabilizer of the complex II, as indicated by our studies using zVAD-fmk. More importantly, future studies that will identify critical targets of RIP1 kinase will further elucidate the signalling mechanisms governing the crosstalk between apoptotic as well as necrotic cell death pathways activated by CD95 or TRAIL-R.

cIAP1 and cIAP2 were originally reported as TRAF-binding proteins⁴⁷. More recently it has been suggested that RIP1 is a direct target of cIAPs^{24, 48} and that the function of cIAPs as constitutive E3 ubiquitin ligases for RIP1 may act independent of the stimulation of death receptors. In particular TRAF2 is a lysine 63 (K63) ubiquitin ligase for RIP1 and K63-RIP1 allows for the further assembly of signalling modules necessary for the activation of NF-κB³¹. In contrast, cIAPs have been suggested to be involved in K63 as well as lysine 48 (K48) ubiquitination of RIP1²⁴. Our own experiments now indicate that one crucial function of cIAPs is to either block RIP1 recruitment to the DISC altogether, or, alternatively, cIAPs may be needed for the rapid degradation of RIP1 within the DISC. Importantly, our experiments cannot distinguish DISC-associated (stimulation-dependent) enrichment of constitutive K48 or K63 ubiquitination of RIP1 by cIAPs as studied by in vitro ubiquitination assays²⁴. When we compared the ubiquitination pattern of RIP1 in the DISC, we detect on long exposures higher molecular weight species of RIP1 in the presence of cIAPs when compared to the absence of cIAPs (compare FIG. 5—long exposures). Our data suggest that constitutively ubiquitinated RIP1 is not recruited to the DISC, or alternatively down-regulate ubiquitinated RIP1 by K48 ubiquitination within the DISC. Future studies using ubiquitination-specific antibodies, as recently described⁴⁹, will be able to kinetically address these points within the different death receptor-induced membrane-associated complexes in more detail. In this context, a recent report studied a mutant of RIP1 that cannot be ubiquitinated at Lys377. These authors showed that it is a non-ubiquitinated form of RIP1 that induces cell death and interacts with caspase-8 at a cytoplasmic complex. In contrast ubiquitinated forms of RIP1 do not induce cell death but require K63 ubiquitination at Lys377 for the protection from cell death, presumably by NF-κB activation⁵⁰.

Why is cFLIP_{L}, but not cFLIP_{S}, able to block cell death whenever IAPs are downregulated? Our data argue for the fact that only cFLIP_{L}, but not cFLIP_{S} represses the efficient formation (or maintenance) of RIP1 in association with caspase-8 within the DISC or complex II altogether. As evident from FIG. 7, complex II contains FADD-RIP1-cFLIP_{S}-caspase-8, and in particular the interaction of RIP1 with caspase-8 is repressed by cFLIP_{L}, but not cFLIP_{S}. These data point to a critical role of the caspase-like domain of cFLIP_{L}. This function could be relevant within the DISC to downregulate RIP1 by cleavage. Alternatively, cFLIP isoforms may also act independent of the death receptor complex, as previously suggested in lymphoid cell⁴⁵. It could be speculated that nonubiquitinated forms of RIP1 could bind to FADD independent of the DISC, subsequently leading to complex II formation and necrotic cell death. Nonetheless, our data clearly indicate that cFLIP_{S} does not have the ability to block the complex II and point to a novel and differential function of different cFLIP isoforms in the absence of cIAPs.

What could be the physiological implication of RIP1 in death receptor signalling? It was recently demonstrated that cell death proceeds in the T cell compartment in a caspase-8 independent manner following stimulation of the T cell receptor. This form of cell death critically required RIP1⁵¹. Taken together, these data argue that in the absence of caspase-8, RIP1 might be critical to eliminate T cells after T cell stimulation and the described proliferative defect of a number of cells in caspase-8 deficient mice⁵² could be caused by a lack of negative regulation of RIP1 by active caspase-8. It is tempting to speculate that this RIP1 degradation occurs within a cytoplasmic caspase-8-FADD-RIP1-complex II. Recent data indicates that a lack of self-processing of caspase-8 does notinterfere with the nonapoptotic functions of caspase-8, whereas apoptosis is compromised⁵³ and argue for a potential chaperone function of caspase-8 at the receptor-independent complex II. Our data further add to these observations and show that although death receptor stimulation principally activates both RIP1 and caspase-8-dependent signals, cIAPs represent crucial negative regulators. RIP1 might act independent of caspase-8 in a number of physiologically and pathophysiologically relevant situations. Future studies that examine conditional mice deficient for both caspase-8 and RIP1 will clarify the physiological role of these two molecules for death receptor and/or TCR-mediated cell death pathways in the immune system in more detail. A number of tumor entities highly express IAPs¹⁶. Based upon our data, it could be possible that cIAPs serve to deviate RIP1-mediated cell death that is possibly associated with necrotic, thus an “immunologically loud” form of cell death. Thus cIAPs may serve an important role to avoid efficient anti tumor immune responses by avoiding death receptor-mediated necrotic cell death¹⁶. More recently, it was shown that caspase-8 negatively regulates cellular transformation⁵⁴ and metastasis⁵⁵. In addition NF-□B is a tumor-promoting transcription factor in a number of cellular systems. Thus cIAPs might also serve an important role during tumorigenesis to shift the death-inducing to a NF-□B inducing function of RIP1. In this context, it will be interesting to determine if tumors that do not overexpress IAPs require an additional loss of RIP1 in order to avoid complex II-mediated cell death or immune activation.

### Material and Methods

Materials. The following antibodies (Abs) were used for Western blot analysis: Abs to caspase-8 (C-15; kindly provided by P. H. Krammer, C-20; Santa Cruz, Delaware Avenue, Calif.), cFLIP (NF-6; Alexis, San Diego, Calif.), FADD and RIP1 (Transduction Laboratories, San Diego, Calif.), CPP32 (kindly provided by H. Mehmet, Merck Frost), caspase-10 (MLB), PARP-1 (clone C-2-10, Biomol), rat Abs to cIAP1 and cIAP2⁵⁶, and β-tubulin (clone 2.1) from Sigma (Saint Louise, Mo., USA). B-actin Abs were from a suitable source. His-FLAG-TRAIL (HF-TRAIL) was produced as recently described¹⁷. For expression of Fc-CD95L we used a construct recently published⁵⁷ (kindly provided by P. Schneider, Epalinges, Switzerland). One unit of Fc-CD95L was determined as a 1:500 dilution of the stock Fc-CD95L supernatant, and one unit/ml of Fc-CD95L supernatant was sufficient to kill 50 percent (LD50) of A375 melanoma cells, as recently described⁵⁸. Ligand-mediated cell death was completely blocked by addition of either soluble TRAIL-R2-Fc protein or CD95-Fc protein, respectively. Horseradish peroxidase (HRP)-conjugated goat anti-rabbit, goat anti rat IgG, goat anti-mouse IgG Abs and HRP-conjugated goat anti-mouse IgG1, IgG2a, IgG2b, and IgG1κ were obtained from Southern Biotechnology Associates (Birmingham, Ala.). TRAIL-R1 (HS 101), TRAIL-R2 (HS 201), mAbs for FACScan analysis of surface receptor expression were used as previously described²⁶ and are available from Alexis (San Diego, Calif.). CD95 Abs (Apo-1 IgG1 and IgG3a) were kindly provided by P. H. Krammer (German Cancer Research Center, Heidelberg, Germany). Cy5-conjugated Annexin V was purchased from Pharmingen (Hamburg, Germany). The IAP inhibitor (compound A) was provided by Tetralogic Pharmaceuticals (Malvern, Pa., USA). Compound A is exemplified in US20060194741, which is incorporated in its entirety by reference herein, and Compound A has the following structure:

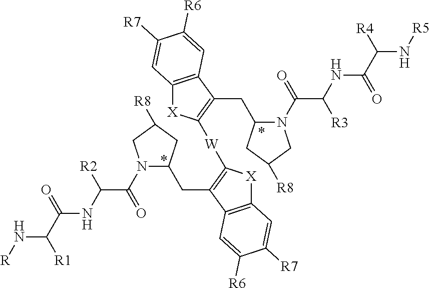

| | | | Ster- | | | | | | | | |
|---|---|---|---|---|---|---|---|---|---|---|---|
| | | | eo- | | | | | | | | |
| | | | chem | | | | | | | | |
| R | R1 | R2 | at * | X | W | R3 | R4 | R5 | R6 | R7 | R8 |
| Me | S- | S- | S | NCH₂CH₂OH | 1,4- | S- | S- | Me | H | F | H |
| | Me | tBu | | | phenyl | tBu | Me | | | | |

Cell culture. The spontaneously transformed keratinocyte line HaCaT and the derived metastatic clone A5-RT3²⁷ were kindly provided by Dr. Petra Boukamp (DKFZ, Heidelberg). MET1 cells⁵⁹ were provided by I. Leigh, Skin Tumor Laboratory, Cancer Research UK, London, UK). Cell lines were exactly cultured as described^{27, 60, 61.}

Retroviral infection. For infection of HaCaT cells, the pCFG5-IEGZ retroviral vector containing cDNA inserts of cFLIP_{L} or cFLIP_{S} was used as previously reported^{62, 62}. Briefly, the amphotrophic producer cell line ΦNX was transfected with 10 μg of the retroviral vectors by calcium phosphate precipitation. Cell culture supernatants containing viral particles were generated by incubation of producer cells with HaCaT medium (DMEM containing 10% FCS) overnight. Following filtration (45 μm filter, Schleicher&Schuell, Dassel, Germany), culture supernatant was added to HaCaT cells seeded in 6 well plates 24 hrs earlier in the presence of 1 μg/ml polybrene. After centrifugation for 3 hrs at 30° C., viral particle containing supernatants were replaced by fresh medium. After 10-14 days zeocin selection of bulk infected cultures, FACS analysis for GFP expression (always >90%, data not shown) and Western blot analysis was performed on polyclonal cells to confirm ectopic expression of the respective molecules. The empty retroviral vector served as control. Aliquots of cells were used for the experiments between passage 2-6 after initial characterization for all subsequent studies.

Stable siRNA expression. We used stable expression of siRNA against cFLIP as recently published⁵⁸. RIP1 siRNA as well as a hyper random sequence not matched by any gene in the NCBI database (HRS)⁶³ were used. For generation of the constructs, cDNA 64-mer oligomers containing RIP1 targeting sequence (nt start position +193: full sequence available upon request) were cloned into the pSuper.retro retroviral vector (pRS) using HindIII and BglII restriction sites. The resulting vectors or control vector containing a not found in the human genome were transfected into the amphotrophic producer cell line exactly as outlined above. The retrovirus-containing supernatant was then used to infect A5RT3 and MET1 cells with HRS shRNA or cFLIP shRNA, respectively. HaCaT cells were infected with HRS and RIP1 shRNA, and infected cells were selected with puromycin (1 μg/ml; Sigma, Taufkirchen, Germany) for 3 days in order to obtain puromycin-resistant bulk infected cultures for further analysis. The respective control constructs served as internal control. FACS analysis of GFP expression (always >90%, data not shown) and Western blot analysis was performed on polyclonal cells to confirm ectopic expression of the respective molecules. Aliquots of cells were used for cytotoxicity assays and biochemical characterization between passage 2 and 6 following the antibiotic selection.

FACScan analysis. For surface staining of TRAIL receptors (TRAIL-R1 and TRAIL-R2) and CD95, cells were trypsinized and 4×10⁵ cells were incubated with monoclonal Abs against TRAIL-R1 TRAIL-R2, CD95, or isotype-matched control IgG for 60 min followed by incubation with biotinylated goat-anti-mouse secondary Abs (BD Pharmingen) and Cy5-Phycoerythrin-labeled streptavidin (Caltag, Burlingame, Calif.) as described³³. For all experiments, 2×10⁴ cells were analyzed by FACScan (Becton Dickinson & Co, San Jose, Calif.).

Western blot analysis. Cell lysates were prepared as described^{17, 58} and 5 μg of total cellular proteins were separated by SDS-PAGE on 4-12% gradient gels (Invitrogen, Karlsruhe, Germany) followed by transfer to nitrocellulose or PVDF membranes. Blocking of membranes and incubation with primary and appropriate secondary Abs were essentially performed as described previously^{33, 62}. Bands were visualized with ECL detection kits (Amersham, Freiburg, Germany).

Cytotoxicity assay. Crystal violet staining of attached, living cells was performed 20-24 h after stimulation with the indicated concentrations of TRAIL or CD95L in 96 well plates in triplicate wells per condition as described³⁷. The optical density (OD) of control cultures was normalized to 100% compared to stimulated cells. For statistical analysis the standard error of mean (SEM) was determined for 3-7 independent experiments of each cell line and stimulatory condition.

Hypodiploidy analysis. Subdiploid DNA content was analyzed as previously performed³³. Briefly, cells were stimulated with the indicated reagents for 8 hrs. Cells were then detached, washed with cold PBS and resuspended in buffer N (Sodium citrate 0.1% (w/v), Triton X 100 0.1% (v/v), PI 50 μg/ml). Cells were kept in the dark at 4° C. for 36-48 hrs and then diploidity was measured by FACScan analysis.

Immunofluorescence microscopy. For detection of nuclear morphology, 5×10⁴ cells of the respective cells were seeded per well in a 12-well plate. Following 24 hrs of incubation for adherence, cells were stimulated as indicated in the Figure legend for 4 or 24 hrs. Subsequently, cells were incubated with Hoechst 33342 (5 μg/ml) for 15 min at 37° C. immediately followed by phase contrast or fluorescence microscopy using a suitable instrument (Leica). Digital images were identically processed using appropriate software.

Annexin V externalization. For the detection of phosphatidylserine externalization, cells were stimulated as indicated in the figure legends. 4 hrs after incubation of cells, trypsinized cells were resuspended in 1× Annexin-V binding buffer (10 mM Hepes, pH7.4, 140 mM NaCl, 2.5 mM CaCl₂) and 2−4×10⁵ cells were subsequently stained with Cy5-conjugated Annexin-V exactly according to the manufacturer (Pharmingen), followed by counterstaining (propidium iodide; 10 μg/ml) for 15 min in the dark at room temperature. For all experiments, 2×10⁴ cells were analyzed by FACScan (Becton Dickinson & Co, San Jose, Calif.).

Colony formation assays. For colony formation assay, 1×10⁴ cells of parental as well as of retrovirally transduced HaCaT cells (HRS, shRNA RIP1, pCFG5-IEGZ retroviral vector and cFLIP_{L} or cFLIP_{S}) was seeded per well in a 24-well plate. After 24 h of incubation adhering cells were either separately prestimulated with IAP inhibitor (100 nM, for 30 min), zVAD-fink (10 μM, for 1 h), Necrostatin-1 (50 μM for 1 h) or in combination of all compounds followed by costimulation with CD95L for 24 hrs. At that time, medium was removed, cells were washed two times with sterile PBS and complete medium was added. Cells were cultured for 3, 5, or 7 days, and subsequently colonies of viable cells were stained by crystal violet as indicated above.

Ligand affinity precipitation of Receptor complexes. For the precipitation of the CD95L DISC, 5×10⁶ cells were used for each condition. Cells were washed once with medium at 37° C. and subsequently preincubated for 1 h with 10 μM zVAD-fmk and, as indicated with 100 nM IAP inhibitor at 37° C. Subsequently cells were treated with 250 units/ml CD95L-Fc for 2 h or, for the unstimulated control, in the absence of ligands. Receptor complex formation was stopped by washing the monolayer four times with ice-cold PBS. Cells were lysed on ice by addition of 2 ml lysis buffer (30 mM Tris-HCl pH 7.5 at 21° C., 120 mM NaCl, 10% Glycerol, 1% Triton X-100, Complete® protease inhibitor cocktail (Roche Molecular Diagnostics, Mannheim, Germany)). After 30 min lysis on ice, the lysates were centrifuged two times at 20,000×g for 5 min and 30 min, respectively, to remove cellular debris. A minor fraction of these clear lysates were used to control for the input of the respective proteins. For the precipitation of the CD95 receptor and stimulation-dependent recruited proteins, Apo-1 IgG3 antibodies (kindly provided by P. H. Krammer) were added to the lysates prepared from non-stimulated as well as stimulated cells to precipitate the receptor-interacting proteins. The levels of receptor precipitated by either ligand affinity precipitation or caspase-8 immunoprecipitation was compared in all experiments by western blotting for CD95 (compare FIG. 5, 7). Receptor complexes were precipitated from the lysates using 40 μl protein G beads (Roche, Mannheim, Germany) for 16-24 hrs on an end-over-end shaker at 4° C. Ligand affinity precipitates were washed 4 times with lysis buffer before the protein complexes were eluted from dried beads by addition of standard reducing sample buffer and boiling at 95° C. Subsequently, proteins were separated by SDS-PAGE on 4-12% NuPAGE gradient gels (Invitrogen, Karlsruhe, Germany) before detection of DISC components by Western blot analysis.

Caspase-8 immunoprecipitation of complex II. Following precipitation of the CD95 DISC, remaining lysates were centrifuged two times at 20,000×g for 5 min. Subsequently 1 μg caspase-8 antibody (C-20, Santa Cruz) were added to all lysates. The caspase-8 containing complexes were precipitated from the lysates by co-incubation with 40 μl protein G beads (Roche, Mannheim, Germany) for 16-24 hrs on an end-over-end shaker at 4° C. Ligand affinity precipitates were washed 4 times with lysis buffer before the protein complexes were eluted from dried beads by addition of standard reducing sample buffer and boiling at 95° C. Subsequently, proteins were separated by SDS-PAGE on 4-12% NuPAGE gradient gels (Invitrogen, Karlsruhe, Germany) before detection of caspase-8-interacting proteins by Western blot analysis. In order to exclude remaining receptor-bound DISC complexes, all caspase-8-interacting complexes were analyzed for the presence of CD95 (compare FIG. 5, 7).

Knockdown of cFLIP. Cell lines were plated into 24 well plates at a density of 35,000 cells per well. Next day, cell lines were transfected with 100 nM control siRNA or siRNA designed to silence expression of cFLIP (L & S isoform). 48 hrs after transfection cells were treated with 100 ng/ml TNFα, 100 nM Smac mimetic, or the combination of both. (The Smac mimetic used herein was a compound other than Compound A.) After an additional 24 hr incubation, all cells were harvested and stained with FITC labeled AnnexinV and Propidium Iodode. Staining was analyzed by flow cytometry.

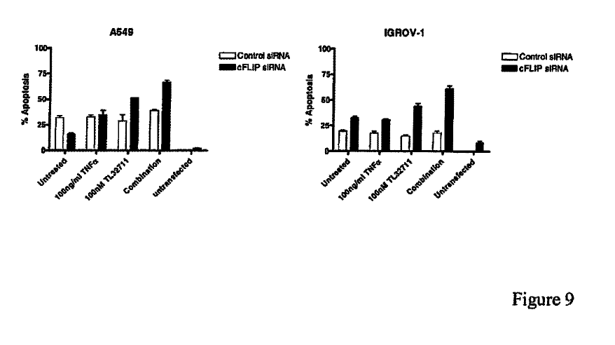

Treatment of cells transfected with control siRNA resulted in no increase of apoptosis under any condition. In contrast, treatment of cells transfected with cFLIP specific siRNAs resulted in some increased sensitivity to the Smac mimetic alone as well as significant sensitization to the combination of the Smac mimetic and TNFα. No significant sensitization to TNFα alone was detected. (FIG. 9.)

These knockdown data show that cFLIP is a cellular mediator of resistance to the combination of Smac mimetics and TNFα. Cell lines which are completely resistant to Smac mimetics and combination of Smac mimetic and TNFα can be sensitized to treatment by the siRNA mediated knockdown of cFLIP. In a clinical setting, levels of cFLIP within a tumor could be used to predict resistance to treatment with Smac mimetic compounds aiding in patient selection.)

### REFERENCES

1. Hanahan, D. & Weinberg, R. A. The hallmarks of cancer. Cell 100, 57-70 (2000).
2. Oltersdorf, T. et al. An inhibitor of Bcl-2 family proteins induces regression of solid tumours. Nature 435, 677-681 (2005).
3. Ashkenazi, A. Targeting the extrinsic apoptosis pathway in cancer. Cytokine Growth Factor Rev. 19, 325-331 (2008).
4. Li, L. et al. A small molecule Smac mimic potentiates TRAIL- and TNFalpha-mediated cell death. Science 305, 1471-1474 (2004).
5. Falschlehner, C., Emmerich, C. H., Gerlach, B., & Walczak, H. TRAIL signalling: Decisions between life and death. Int. J. Biochem. Cell Biol. 39, 1462-1475 (2007).
6. Peter, M. E. & Krammer, P. H. The CD95 (APO-1/Fas) DISC and beyond. Cell Death. Differ. 10, 26-35 (2003).
7. Johnstone, R. W., Frew, A. J., & Smyth, M. J. The TRAIL apoptotic pathway in cancer onset, progression and therapy. Nat. Rev. Cancer 8, 782-798 (2008).
8. Meier, P. & Vousden, K. H. Lucifer's labyrinth—ten years of path finding in cell death. Mol. Cell. 28, 746-754 (2007).
9. Micheau, O. & Tschopp, J. Induction of TNF receptor I-mediated apoptosis via two sequential signaling complexes. Cell 114, 181-190 (2003).
10. Lavrik, I. N. et al. CD95 stimulation results in the formation of a novel death effector domain protein-containing complex. J Biol Chem 283, 26401-26408 (2008).
11. Varfolomeev, E. et al. Molecular determinants of kinase pathway activation by apo2 ligand/tumor necrosis factor related apoptosis-inducing ligand. J. Biol. Chem. (2005).
12. Kroemer, G. et al. Classification of cell death: recommendations of the Nomenclature Committee on Cell Death 2009. Cell Death. Differ. (2008).
13. Casares, N. et al. Caspase-dependent immunogenicity of doxorubicin-induced tumor cell death. J Exp. Med. 202, 1691-1701 (2005).
14. Kazama, H. et al. Induction of immunological tolerance by apoptotic cells requires caspase-dependent oxidation of high-mobility group box-1 protein. Immunity. 29, 21-32 (2008).
15. Ashkenazi, A., Holland, P., & Eckhardt, S. G. Ligand-based targeting of apoptosis in cancer: the potential of recombinant human apoptosis ligand 2/Tumor necrosis factor-related apoptosis-inducing ligand (rhApo2L/TRAIL). J. Clin. Oncol. 26, 3621-3630 (2008).
16. Wright, C. W. & Duckett, C. S. Reawakening the cellular death program in neoplasia through the therapeutic blockade of IAP function. J. Clin. Invest 115, 2673-2678 (2005).
17. Diessenbacher, P. et al. NF-kappaB inhibition reveals differential mechanisms of TNF versus TRAIL-induced apoptosis upstream or at the level of caspase-8 activation independent of cIAP2. J Invest Dermatol 128, 1134-1147 (2008).
18. Wang, C. Y., Mayo, M. W., Korneluk, R. G., Goeddel, D. V., & Baldwin, A. S., Jr. NF-kappaB antiapoptosis: induction of TRAF1 and TRAF2 and c-IAP1 and c-IAP2 to suppress caspase-8 activation. Science 281, 1680-1683 (1998).
19. Vogler, M. et al. Targeting XIAP bypasses Bcl-2-mediated resistance to TRAIL and cooperates with TRAIL to suppress pancreatic cancer growth in vitro and in vivo. Cancer Res 68, 7956-7965 (2008).
20. Gaither, A. et al. A Smac mimetic rescue screen reveals roles for inhibitor of apoptosis proteins in tumor necrosis factor-alpha signaling. Cancer Res 67, 11493-11498 (2007).
21. Petersen, S. L. et al. Autocrine TNFalpha signaling renders human cancer cells susceptible to Smac-mimetic-induced apoptosis. Cancer Cell 12, 445-456 (2007).
22. Vince, J. E. et al. IAP antagonists target cIAP1 to induce TNFalpha-dependent apoptosis. Cell 131, 682-693 (2007).
23. Varfolomeev, E. et al. IAP antagonists induce autoubiquitination of c-IAPs, NF-kappaB activation, and TNFalpha-dependent apoptosis. Cell 131, 669-681 (2007).
24. Bertrand, M. J. et al. cIAP1 and cIAP2 facilitate cancer cell survival by functioning as E3 ligases that promote RIP1 ubiquitination. Mol. Cell. 30, 689-700 (2008).
25. Lotze, M. T. et al. The grateful dead: damage-associated molecular pattern molecules and reduction/oxidation regulate immunity. Immunol. Rev. 220, 60-81 (2007).
26. Leverkus, M. et al. Proteasome inhibition results in TRAIL sensitization of primary keratinocytes by removing the resistance-mediating block of effector caspase maturation. Mol. Cell. Biol. 23, 777-790 (2003).
27. Mueller, M. M. et al. Tumor progression of skin carcinoma cells in vivo promoted by clonal selection, mutagenesis, and autocrine growth regulation by granulocyte colony-stimulating factor and granulocyte-macrophage colony-stimulating factor. Am. J. Pathol. 159, 1567-1579 (2001).
28. Walczak, H. & Haas, T. L. Biochemical analysis of the native TRAIL death-inducing signaling complex. Methods Mol. Biol. 414, 221-239 (2008).
29. Holler, N. et al. Fas triggers an alternative, caspase-8-independent cell death pathway using the kinase RIP as effector molecule. Nat. Immunol. 1, 489-495 (2000).
30. Matsumura, H. et al. Necrotic death pathway in Fas receptor signaling. J Cell Biol 151, 1247-1256 (2000).
31. Festjens, N., Vanden, B. T., & Vandenabeele, P. RIP1, a kinase on the crossroads of a cell's decision to live or die. Cell Death. Differ. 14, 400-410 (2007).
32. Degterev, A. et al. Identification of RIP1 kinase as a specific cellular target of necrostatins. Nat. Chem. Biol 4, 313-321 (2008).
33. Wachter, T. et al. cFLIPL inhibits tumor necrosis factor-related apoptosis-inducing ligand-mediated NF-kappaB activation at the death-inducing signaling complex in human keratinocytes. J. Biol. Chem. 279, 52824-52834 (2004).
34. Krueger, A., Baumann, S., Krammer, P. H., & Kirchhoff, S. FLICE-inhibitory proteins: regulators of death receptor-mediated apoptosis. Mol. Cell. Biol. 21, 8247-8254 (2001).
35. Budd, R. C., Yeh, W. C., & Tschopp, J. cFLIP regulation of lymphocyte activation and development. Nat. Rev. Immunol. 6, 196-204 (2006).
36. Yu, J. W. & Shi, Y. FLIP and the death effector domain family. Oncogene 27, 6216-6227 (2008).
37. Leverkus, M. et al. Regulation of tumor necrosis factor-related apoptosis-inducing ligand sensitivity in primary and transformed human keratinocytes. Cancer Res. 60, 553-559 (2000).
38. Krueger, A., Schmitz, I., Baumann, S., Krammer, P. H., & Kirchhoff, S. Cellular FLICE-inhibitory protein splice variants inhibit different Steps of caspase-8 activation at the CD95 death-inducing signaling complex. J. Biol. Chem. 276, 20633-20640 (2001).
39. Kelliher, M. A. et al. The death domain kinase RIP mediates the TNF-induced NF-kappaB signal. Immunity. 8, 297-303 (1998).
40. Vercammen, D. et al. Dual signaling of the Fas receptor: initiation of both apoptotic and necrotic cell death pathways. J Exp. Med. 188, 919-930 (1998).
41. Vanden, B. T. et al. Differential signaling to apoptotic and necrotic cell death by Fas-associated death domain protein FADD. J Biol Chem 279, 7925-7933 (2004).
42. Meurette, O. et al. TRAIL induces receptor-interacting protein 1-dependent and caspase-dependent necrosis-like cell death under acidic extracellular conditions. Cancer Res 67, 218-226 (2007).
43. Wang, L., Du, F., & Wang, X. TNF-alpha induces two distinct caspase-8 activation pathways. Cell 133, 693-703 (2008).
44. Boatright, K. M., Deis, C., Denault, J. B., Sutherlin, D. P., & Salvesen, G. S. Activation of caspases-8 and -10 by FLIP(L). Biochem. J 382, 651-657 (2004).
45. Golks, A., Brenner, D., Krammer, P. H., & Lavrik, I. N. The c-FLIP-NH2 terminus (p22-FLIP) induces NF-kappaB activation. J Exp. Med. 203, 1295-1305 (2006).
46. Kataoka, T. The caspase-8 modulator c-FLIP. Crit. Rev Immunol 25, 31-58 (2005).
47. Rothe, M., Pan, M. G., Henzel, W. J., Ayres, T. M., & Goeddel, D. V. The TNFR2-TRAF signaling complex contains two novel proteins related to baculoviral inhibitor of apoptosis proteins. Cell 83, 1243-1252 (1995).
48. Park, S. M., Yoon, J. B., & Lee, T. H. Receptor interacting protein is ubiquitinated by cellular inhibitor of apoptosis proteins (c-IAP 1 and c-IAP2) in vitro. FEBS Lett. 566, 151-156 (2004).
49. Newton, K. et al. Ubiquitin chain editing revealed by polyubiquitin linkage-specific antibodies. Cell 134, 668-678 (2008).
50. O'Donnell, M. A., Legarda-Addison, D., Skountzos, P., Yeh, W. C., & Ting, A. T. Ubiquitination of RIP1 regulates an NF-kappaB-independent cell-death switch in TNF signaling. Curr. Biol 17, 418-424 (2007).
51. Ch'en, I. L. et al. Antigen-mediated T cell expansion regulated by parallel pathways of death. Proc. Natl. Acad. Sci. U.S. A (2008).
52. Kang, T. B. et al. Caspase-8 serves both apoptotic and nonapoptotic roles. J Immunol. 173, 2976-2984 (2004).
53. Kang, T. B. et al. Mutation of a self-processing site in caspase-8 compromises its apoptotic but not its nonapoptotic functions in bacterial artificial chromosome-transgenic mice. J Immunol. 181, 2522-2532 (2008).
54. Krelin, Y. et al. Caspase-8 deficiency facilitates cellular transformation in vitro. Cell Death. Differ. 15, 1350-1355 (2008).
55. Stupack, D. G. et al. Potentiation of neuroblastoma metastasis by loss of caspase-8. Nature 439, 95-99 (2006).
56. Silke, J. et al. Determination of cell survival by RING-mediated regulation of inhibitor of apoptosis (IAP) protein abundance. Proc. Natl. Acad. Sci. U.S. A 102, 16182-16187 (2005).
57. Bossen, C. et al. Interactions of tumor necrosis factor (TNF) and TNF receptor family members in the mouse and human. J Biol Chem 281, 13964-13971 (2006).
58. Geserick, P. et al. Suppression of cFLIP is sufficient to sensitize human melanoma cells to TRAIL- and CD95L-mediated apoptosis. Oncogene 27, 3211-3220 (2008).
59. Popp, S. et al. Genetic characterization of a human skin carcinoma progression model: from primary tumor to metastasis. J. Invest Dermatol 115, 1095-1103 (2000).
60. Boukamp, P. et al. Normal keratinization in a spontaneously immortalized aneuploid human keratinocyte cell line. J. Cell Biol. 106, 761-771 (1988).
61. Proby, C. M. et al. Spontaneous keratinocyte cell lines representing early and advanced stages of malignant transformation of the epidermis. Exp. Dermatol 9, 104-117 (2000).
62. Leverkus, M. et al. TRAIL-induced apoptosis and gene induction in HaCaT keratinocytes: differential contribution of TRAIL receptors 1 and 2. J. Invest Dermatol 121, 149-155 (2003).
63. Vogler, M., Durr, K., Jovanovic, M., Debatin, K. M., & Fulda, S. Regulation of TRAIL-induced apoptosis by XIAP in pancreatic carcinoma cells. Oncogene 26, 248-257 (2007).
64. Goto et. al., Porcine (sus scrofaa) Cellular FLICE-like Inhibitory Protein (cFLIP): Molecular Cloning and Comparison with the Human and Murine cFLIP, J. of Reproduction and Development, 50, 549-555 (2004).

1. A method of determining sensitivity of human or non-human animal cells to an IAP antagonist comprising determining if the cells can express cFLIP_{L}, whereby cells that can express cFLIP_{L} are resistant to an IAP antagonist.

2. The method of claim 1 wherein sensitivity of the cells to an IAP antagonist in combination with a TRAIL receptor agonist, a CD95 receptor agonist or a TNFα receptor agonist is determined.

3. The method of claim 2 wherein the TRAIL receptor agonist is TRAIL, the CD95 receptor agonist is CD95L (FasL), and the TNFα receptor is TNFα.

4. The method of claim 1 wherein the cells are from a biopsy sample.

5. The method of claim 1 wherein the potential for expression of the cFLIP_{L} gene is assayed by a method selected from the group consisting of:
(a) determining the presence of cFLIP_{L} mRNA in the cell, and
(b) determining the presence of cFLIP_{L} in the cell.

6. The method of claim 1 wherein the cells comprise a cell line.

7. The method of claim 1 wherein the cells are selected from the group consisting of tumor cells and cells which abnormally proliferate in an autoimmune disorder.

8. A method of determining sensitivity of human or non-human animal cells to an IAP antagonist comprising determining if the cells can express cFLIP_{S}, whereby cells that can express cFLIP_{S} are sensitive to an IAP antagonist.

9. The method of claim 8 wherein sensitivity of the cells to an IAP antagonist in combination with a TRAIL receptor agonist, a CD95 receptor agonist or a TNFα receptor agonist is determined.

10. The method of claim 9 wherein the TRAIL receptor agonist is TRAIL, the CD95 receptor agonist is CD95L, and the TNFα receptor is TNFα.

11. The method of claim 8 wherein the cells are from a biopsy sample.

12. The method of claim 8 wherein the potential for expression of the cFLIP_{S} gene is assayed by a method selected from the group consisting of:
(a) determining the presence of cFLIP_{S} mRNA in the cell, and
(b) determining the presence of cFLIP_{S} in the cell.

13. The method of claim 8 wherein the cells comprise a cell line.

14. The method of claim 8 wherein the cells are selected from the group consisting of tumor cells and cells which abnormally proliferate in an autoimmune disorder.

15. A method of treating a patient suffering a proliferative disorder that comprises:
(a) determining the sensitivity of some or all of the proliferative cells to treatment with an IAP antagonist by determining if the cells can express cFLIP_{L} or cFLIP_{S}, whereby cells that can express cFLIP_{S} are sensitive to an IAP antagonist. and
(b) if the cells can express cFLIP_{S}, then treating the cells with an IAP antagonist or
(c) if the cells can cFLIP_{L}, then treating the cells with an agent other than or additional to an IAP antagonist.

16. The method of claim 15 wherein sensitivity of the cells to an IAP antagonist in combination with a TRAIL receptor agonist, a CD95 receptor agonist or a TNFα receptor agonist is determined.

17. The method of claim 16 wherein the TRAIL receptor agonist is TRAIL, the CD95 receptor agonist is CD95L (FasL), and the TNFα receptor is TNFα.

18. The method of claim 15 wherein the cells are from a biopsy sample.

19. The method of claim 15 wherein the potential for expression of the cFLIP_{L} gene is assayed by a method selected from the group consisting of:
(a) determining the presence of cFLIP_{L} mRNA in the cell and
(b) determining the presence of cFLIP_{L} in the cell.

20. The method of claim 15 wherein the cells comprise a cell line.

21. The method of claim 15 wherein the cells are selected from the group consisting of tumor cells and cells which abnormally proliferate in an autoimmune disorder.

22. A method of inducing apoptosis, comprising:
(a) assaying cells of a cell population to determine expression of cFLIPs; and
(b) contacting the cell population with an IAP antagonist if cFLIP_{S} protein expression is detected.

23. A method of screening cancer patients for those who could benefit from treatment with an IAP antagonist, comprising:
(a) assaying abnormally proliferating cells obtained from a patient for cFLIP_{S} gene/protein expression;
(b) selecting those patients in whom the proliferating cells can express cFLIP_{S} for treatment with an IAP antagonist.

24. A method of selecting patients suffering a proliferative disorder for inclusion in a clinical trial of an IAP antagonist that comprises:
(a) assaying abnormally proliferating cells obtained from each patient for cFLIP_{S} gene/protein expression;
(b) including in the clinical trial those patients in whom the proliferating cells can express cFLIP_{S}.
